(19) United States
(12) Patent Application Publication
(10) Pub. No.: US 2016/0324863 A1
(43) Pub. Date: Nov. 10, 2016

(54) TARGETED CANCER TREATMENT BY HSP90 INHIBITORS GANETESPIB AND NVP-AUY922

(71) Applicant: SYNTA PHARMACEUTICALS CORP., Lexington, MA (US)

(72) Inventors: Jaime Acquaviva, Ashland, MA (US); David Proia, Newton, MA (US)

(73) Assignee: Synta Pharmaceuticals Corp., Lexington, MA (US)

(21) Appl. No.: 15/111,977

(22) PCT Filed: Jan. 16, 2015
(86) PCT No.: PCT/US2015/011803

§ 371 Date: Jul. 15, 2016

Related U.S. Application Data
(60) Provisional application No. 61/994,196, filed on May 16, 2014.
(60) Provisional application No. 61/928,601, filed on Jan. 17, 2014.

(51) Int. Cl. A61K 31/5377; C12Q 1/68; A61K 45/06; A61K 31/4196; A61K 31/506

(52) U.S. Cl. CPC A61K 31/5377; A61K 31/4196; A61K 31/506; A61K 45/06; C12Q 1/6886; C12Q 2600/106

(57) ABSTRACT

Methods of treating certain cancers with ganetespib or NVP-AUY922 are disclosed. Also provided are methods of treating cancer wherein the cancer has an FGFR3 mutation and/or a low level expression of UGT1A, and/or a low level expression of UGT1A9 and/or UGT1A10. Also provided are methods of treating cancer with a mutation in FGFR3 and/or a low level expression of UGT1A, and/or a low level expression of UGT1A9 and/or UGTIA10, with ganetespib or NVP-AUY922, in combination with an FGFR3 inhibitor such as BGJ398 and AZD4547.

### CROSS-REFERENCE TO RELATED PATENTS

This application claims the benefit of priority to U.S. Provisional Patent Application No. 61/994,196, filed on May 16, 2014 and U.S. Provisional Patent Application No. 61/928,601 filed on Jan. 17, 2014. The contents of the above applications are incorporated by reference herein.

### BACKGROUND OF THE INVENTION

Cancer is a group of diseases characterized by dysregulation of cell differentiation and proliferation and, in advanced stages, spreads to other areas of the body including vital organs and bone. If not brought under control, these diseases can be fatal.

Although tremendous advances have been made in elucidating the genomic abnormalities that cause malignant cancer cells and the availability of multiple therapeutic regimens to treat cancer, currently available chemotherapy still remains unsatisfactory, and the prognosis for the majority of patients diagnosed with cancer remains dismal. Furthermore, many patients do not even respond to any treatments. Of those that do respond to standard therapies, the effect is usually short-lived as resistance develops to the initial therapeutic regimens. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it appears increasingly unlikely that a therapeutic agent that acts on one molecular target will be fully effective in curing a patient who has cancer.

More particularly, bladder cancer represents the fifth most common malignancy worldwide and a major cause of cancer related morbidity and death. A vast majority of patients, over 70%, present with non-muscle invasive bladder cancer (NMIBC), previously referred to as ‘superficial’ bladder cancer. Despite their relatively benign nature, these tumors have a characteristically high risk of recurrence, which presents an immense challenge for the clinical management and ongoing surveillance of patients, often over many years. The remaining cases are diagnosed as muscle invasive bladder cancer (MIBC), a more aggressive stage of the disease that is associated with a higher risk of metastasis and for which the 5-year survival rates are only around 50%. Indeed, progression to metastatic bladder cancer, whether from recurrent NMIBC or MIBC, results in significantly poorer survival outcomes and is generally considered incurable. In contrast to other malignancies, advances in managing and treating bladder cancer have been limited. Consequently, there exists a considerable unmet need for novel therapeutic approaches to improve patient outcomes in this disease indication.

### SUMMARY OF THE INVENTION

Certain resorcinol-derived Hsp90 inhibitors are demonstrated herein to be particularly effective in treating humans with cancer. These Hsp90 inhibitors are shown to be particularly effective in treating certain specific types of cancer, including cancers having a mutation in FGFR3; cancers with low level expression of UTG1A; and/or low level expression of UGT1A9 and/or UGT1A10. The particular dosing regimens disclosed herein demonstrate potency against certain specific types of cancer, while showing optimal treatment effects and minimal toxic side effects.

Methods of treating cancer in a subject, comprising administering ganetespib or NVP-AUY922, or pharmaceutically acceptable salts or tautomers thereof, wherein the cancer has a mutation in FGFR3 are disclosed. In an embodiment, these methods also include using ganetespib or NVP-AUY922 in treating a subject with cancer having a mutation in FGFR3 and/or a low level expression of UGT1A, and/or low level expression of UGT1A9 and/or UGT1A10. In another embodiment, the method further includes using ganetespib or NVP-AUY922 in treating a subject with cancer having a mutation in FGFR3 and/or having low level expression of UGT1A and/or low level expression of UGT1A9 and/or UGT1A10.

In certain embodiments, the cancer may be multiple myeloma, bladder cancer, cervical carcinoma, urothelial carcinoma, colorectal cancer, or solid tumor.

In certain embodiments, methods disclosed herein further include identifying a subject as having a cancer with a mutation in FGFR3. In certain embodiments, methods of the invention also include identifying the level of the expression of UGT1A in a subject with cancer. In certain embodiments, methods disclosed herein also include identifying the level of expression of UGT1A9 and/or UGT1A10 in a subject with cancer. In certain embodiments, methods disclosed herein further include identifying a subject as having a cancer with a mutation in FGFR3 and with a low level expression of UGT1A for treatment. In certain embodiments, methods disclosed herein further include identifying a subject as having a cancer with a mutation in FGFR3 and with a low level expression of UGT1A9 and/or UGT1A10 for treatment. In certain embodiments, methods disclosed herein further include identifying a subject as having a cancer with a mutation in FGFR3 and with a high level expression of UGT1A for a different treatment regimen. In certain embodiments, methods disclosed herein further include identifying a subject as having a cancer with a mutation in FGFR3 and with a high level expression of UGT1A9 and/or UGT1A10 for a different treatment regimen.

In certain embodiments, the methods include administration of ganetespib or a pharmaceutically acceptable salt or a tautomer thereof, at a dose of 2 mg/m² to 260 mg/m², or in any amount falling within that range.

In certain embodiments, the methods include administration of NVP-AUY922 or a pharmaceutically acceptable salt or a tautomer thereof, at a dose of 0.5 mg/kg to 200 mg/kg, or in any amount falling within that range.

In certain embodiments, the invention further includes administering an FGFR3 inhibitor including BGJ398 and AZD4547. In certain embodiments, the invention further includes administering one or more additional anticancer agents, such as BEZ-235, AZD6244, AZD8055, SN-38, gemcitabine, camptothecin, docetaxel, cisplatin, oxaliplatin, crizotinib, paclitaxel, trastuzumab, or pemetrexed.

In certain embodiments, the levels of UGT1A and UGT1A9 and UGT1A10, and mutations are detected in a subject sample, e.g., a tumor sample, and compared to an appropriate control.

The invention provides methods for identifying a subject for treatment with ganetespib or NVP-AUY922, including providing a subject sample from the subject, determining the level of UGT1A and/or UGT1A9 and/or UGT1A10 in a tumor from the subject (advantageously in vitro), and determining (advantageously in vitro) if the subject has a tumor with a mutation in FGFR3 wherein a low level expression of UGT1A and/or UGT1A9 and/or UGT1A10 in the sample and a mutation in FGFR3, indicates the subject is likely to respond to therapy with ganetespib or NVP-AUY922.

The invention also provides kits to practice the methods. For example, a kit can include an instruction for administration of ganetespib or NVP-AUY922 to a subject having cancer with a low level expression of UGT1A and/or UGT1A9 and/or UGT1A10 and/or with a mutation in FGFR3. Such a kit can also include information on measuring the level of the expression of UGT1A and/or UGT1A9 and/or UGT1A10 and/or on determining a mutation in FGFR3.

In an embodiment, the method includes treating, managing, or ameliorating cancer or one or more symptoms thereof, wherein the cancer has a mutation in FGFR3 and/or with a low level expression of UGT1A and/or with a low level expression of UGT1A9 and/or UGT1A10, comprising administering to a subject in need thereof an effective amount of ganetespib or NVP-AUY922, or pharmaceutically acceptable salts or tautomers thereof, in combination with an FGFR3 inhibitor. In an embodiment, the method includes treating, managing, or ameliorating multiple myeloma, bladder cancer, cervical carcinoma, urothelial carcinoma, or solid tumor, or one or more symptoms thereof, comprising administering to a subject in need thereof an effective amount of ganetespib or NVP-AUY922, or a pharmaceutically acceptable salt thereof, in combination with an FGFR3 inhibitor.

In an embodiment, the method includes treating, managing, or ameliorating cancer, or one or more symptoms thereof, wherein the cancer has a mutation in FGFR3 and/or with a low level expression of UGT1A and/or with a low level expression of UGT1A9 and/or UGT1A10, comprising administering to a subject in need thereof an effective amount of ganetespib, or NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, or solid tumor, or one or more symptoms thereof, comprising administering to a subject in need thereof an effective amount of ganetespib or NVP-AUY922, or pharmaceutically acceptable salts or tautomers thereof, in combination with BGJ398.

### BRIEF DESCRIPTION OF THE DRAWINGS

FIG. 1 shows the viability of RT112 cells when treated with increasing concentrations of ganetespib and BGJ398, respectively after 72 hours.

FIG. 2 shows the viability of RT112 cells when treated with graded concentrations of ganetespib, as indicated. Caspase 3/7 activity was measured at 24 hours and viability was assessed at 72 hours post-treatment (“RLU”: relative luminescence units).

FIG. 3 shows the expression levels of FGFR3-TAAC3, phosphorylated FGFR3, ERK and AKT (p-FGFR, p-ERK, p-AKT) when the RT112 cells were exposed to increasing concentrations of ganetespib, and BGJ398, respectively, at 100 nM, for 24 hours. BIM induction was assessed by immunoblotting; “GAPDH” means glyceraldehyde-3-phosphate dehydrogenase.

FIG. 4 shows the expression levels of FGFR3-TAAC3 and others when RT112 cells were treated with 200 nM ganetespib and harvested at 2, 4, 6 and 24 hours, respectively. Lysates were immunoblotted with the indicated antibodies. “V”: vehicle control at 24 hour time point.

FIG. 5 shows the 72-hour combination viability assay with ganetespib and BGJ398 in RT112 cells. Percentages represent the amount of cell death observed at each of the corresponding dose levels.

FIG. 6 shows the results of treatment of mice bearing established RT112 xenografts (n=8 mice/group) when dosed i.v. with ganetespib (150 mg/kg) once-weekly and BGJ398 (10 mg/kg) administered p.o. 7×/week, alone and in combination. Percent T/C values are indicated to the right of each growth curve, and the error bars are the SEM.

FIG. 7 shows the results of treatment of mice bearing established RT112 s xenografts (n=3 animals/group) when treated with either vehicle or ganetespib (150 mg/kg) for 24 and 72 hours. Tumors were resected and the relative expression levels of the indicated signaling proteins determined by phosphoprotein array analysis.

FIG. 8 shows the viability of RT4 cells when treated with increasing concentrations of ganetespib, AUY922, 17-AAG and 17-DMAG at 72 hours.

FIG. 9 shows immunoblots indicating the expression levels of wild type FGFR3 and the FGFR3-TAAC3 fusion protein (higher MW bands) of RT4 cells when exposed to graded concentrations of 17-AAG or 17-DMAG, as indicated (“V:” vehicle control.)

FIG. 10 shows immunoblots indicating the levels of p-ERK, HER2, CDC2 and BIM of RT4 cells when treated with 17-DMAG or 17-AAG, as indicated, for 24 hours.

FIG. 11 shows the expression levels of RT4 cells when exposed to increasing concentrations of ganetespib (0.1-1 μM) for 24 hours (lysates were immunoblotted with the indicated antibodies. “V”: vehicle control)

FIG. 12 demonstrates the viability of the SW780 cells when treated with increasing concentrations of ganetespib, AUY922, 17-AAG and 17-DMAG at 72 hours.

FIG. 13 shows the expression levels of SW780 cells when treated with ganetespib, AUY922 or 17-AAG at the indicated concentrations for 24 hours (lysates were immunoblotted with the indicated antibodies. “V”: vehicle control.)

FIG. 14 shows the expression levels of SW780 cells when exposed to increasing concentrations of ganetespib (0.1-5 μM) for 24 hours (lysates wert immunoblotted with the indicated antibodies).

FIG. 15 shows the intracellular drug concentrations of the respective Hsp90 inhibitors as a function of dose and time when RT112 and SW780 cells were treated with increasing concentrations of ganetespib or 17-DMAG (10-1000 nM), respectively, for 1 and 24 hours, as indicated.

FIG. 16 shows the intracellular concentration of ganetespib and its glucuronidated metabolites, as well as drug concentrations in the media (determined by LC-MS/MS. glu-ganetespib: total combined concentration of the metabolites). The RT112 and SW780 cells were cultured in 1 μM ganetespib. Cellular lysates were prepared and culture media was collected at 15 minutes, 1, 4, 8, and 24 hours, respectively.

FIG. 17 shows UGT1A expression of bladder cancer cells when treated with 100 nM (RT112) or 1000 nM (RT4, SW780) ganetespib, or vehicle, for 24 hours.

FIG. 18 shows a scatter plot of differential metabolism gene expression between RT112 and SW780 cell lines using microarray analysis. The axes of the scatter plot are the normalized signal values (ratio scale); outer parallel lines indicate a twofold-change in gene expression threshold. Each spot represents an individual gene, and members of the UGT1A family of enzymes are presented as red diamonds.

FIG. 19 shows the UGT1A9 expression of cellular lysates prepared from UGT1A9-transfected 293T cells (positive control), SW780, RT4, RT112 and 97-7 bladder cancer cell lines.

FIG. 20 shows the IC50 values of ganetespib for 11 colorectal cancer cell lines (blue columns), and the corresponding expression levels of pan-UGT1A obtained through microarray analysis and re-determined by reverse transcription and quantitative PCR (red columns).

FIG. 21 shows the immunoblots indicating the UGT1A protein levels of the cell lines as depicted in FIG. 20. GAPDH detection serves as a loading control.

FIG. 22 is a graph showing the distribution of UGT1A mRNA expression levels in primary colorectal tumors. Microarray hybridization datasets from 217 rectal carcinomas were obtained. The mean hybridization intensities from three probes corresponding to UGT1A (log 2 scale) were normalized according to their deviation from the overall mean intensity of all tumors. The distribution of UGT1A levels was determined in the same way for the 11 CRC cell lines as depicted in FIG. 20.

FIG. 23 shows the ICso values of 17AAG for 11 colorectal cancer cell lines as depicted in FIG. 20 (green columns), and the corresponding expression levels of pan-UGT1A obtained through microarray analysis and re-determined by reverse transcription and quantitative PCR (red columns).

FIG. 24 shows ICso values for ganetespib, AUY922 and 17AAG/17DMAG, respectively in indicated colon cancer cell lines.

FIG. 25 shows quantitative RT-PCR (left) and immunoblots (right) indicating the reduction of the UGT1A mRNA and protein levels by siRNA-mediated knockdown.

FIG. 26 shows the proliferation profile of the HT29 cells upon UGT1A knockdown in response to ganetespib. Ganetespib was added 24 hours after siRNA transfection to the cells at the indicated concentrations, and ganetespib-containing medium was refreshed every 24 hours. Cell confluence was determined by quantitative light microscopy (Celligo) 72 hours after first addition of the drug.

FIG. 27 shows the cell viability upon UGT1A knockdown at indicated ganetespib concentrations. Upon treatment as depicted in FIG. 26, cell viability was determined by assessing the ATP concentration in cell lysates using a luciferase assay.

FIG. 28 is a graph showing the UGT1A gene clusters by selective RT-PCR analysis in comparison to a primer pair that amplifies all isoforms, revealing that the cluster composed of isoforms 7, 8, 9 and 10 is expressed at a particularly high level in the ganetespib-resistant cell line HT29. Thus, isoform 10 was chosen for overexpression in FIG. 29.

FIG. 29 shows the confluence at indicated ganetespib concentrations determined 72 hours later by Celligo.

FIG. 30 shows the cell viability at indicated ganetespib concentrations determined by ATP quantification via luciferase as depicted in FIG. 27.

FIG. 31 shows immunoblots indicating the expression levels of the Hsp90 client proteins AKT and Wee1 for ganetespib-sensitive (SW480, HCT116) and resistant (SW1463, HT29) cells each treated with ganetespib.

FIG. 32 shows immunoblots indicating the expression levels of the Hsp90 client proteins for the indicated cells treated and analyzed as depicted in FIG. 31, but 17-AAG was used instead of ganetespib.

FIG. 33 shows immunoblots indicating the expression levels of the Hsp90 client proteins of the HT29 cells first transfected with siRNA for 24 hours, followed by incubation with ganetespib for 48 hours at the indicated concentrations. To avoid complete ganetespib turnover, ganetespib-containing media were renewed every 8 hours during the incubation period.

FIG. 34 shows immunoblots indicating the expression levels of the Hsp90 client proteins of the HCT116 and SW480 cells transfected with the overexpression vector and (3-Galactosidase vector as control, and treated with the indicated concentrations of ganetespib for 48 hours before harvesting. GAPDH was used as loading control.

FIG. 35 is a graph showing the concentrations of ganetespib and its glucuronidated metabolites at indicated time points after HCT116, SW480, SW1463 and HT29 cells were treated with 1 μM ganetespib. Cell lysates were prepared at 5, 15, 30, 60 and 480 minutes, and the intracellular concentration of ganetespib and its glucuronidated metabolites were determined by LC-MS/MS.

FIG. 36 is a graph showing the concentrations of ganetespib and its glucuronidated metabolites at indicated time points after HCT116, SW480, SW1463 and HT29 cells were treated by 1 μM ganetespib. Culture media was collected at 5, 15, 30, 60 and 480 minutes, and the concentrations ganetespib and its glucuronidated metabolites in the media were determined by LC-MS/MS.

### DETAILED DESCRIPTION OF THE INVENTION

### Definitions

Fibroblast growth factor receptor 3 (FGFR3) belongs to the type III receptor-tyrosine kinase family that responds to fibroblast growth factor (FGF), and negatively regulates bone formation in mammals. FGF binds to the extracellular domain of FGFR3 and results in receptor oligomerization, which leads to autophosphorylation at tyrosine residues in the cytoplasmic domains. The phosphorylated tyrosine residues are required for either stimulation of the intrinsic catalytic activity or activation of downstream signaling pathways by providing docking sites for SH2 domain containing signaling components. FGFR3 is oncogenic when activated by FGF ligands or by activating mutations. Overexpression of FGFR3 wild-type or activated FGFR3 TDII mutant transforms murine B9 myeloma cells to IL-6-independent growth. The activated form of FGFR3 induces transformation of NIH3T3 cells that are tumorigenic when injected into nude mice. Moreover, in a murine bone marrow transplantation (BMT) model, mice transplanted with bone marrow cells transduced by retroviral vectors, encoding the activated FGFR3 TDII mutant, rapidly develop a lethal pre-B cell lymphoma.

FGFR3 is a member of a structurally related family of tyrosine kinase receptors (FGFR1-4) that orchestrate a diverse variety of cellular activities, including proliferation, differentiation, and survival. Ligand binding promotes receptor dimerization, transphosphorylation of key tyrosine residues, and recruitment of adaptor proteins—ultimately leading to the activation of multiple downstream signaling cascades, including PI3K/AKT, RAS/MAPK, STATs, and phospholipase Cγ (PLCγ). Somatic mutation of the FGFR3 gene is one of the most frequent genetic alterations seen in bladder cancer, occurring in around 75% of all cases of NMIBC. Interestingly, most of the missense mutations identified in bladder tumors are identical to germline gain-of-function mutations responsible for autosomal dominant human skeletal disorders and dwarfism syndromes. FGFR3 mutations are less prevalent in muscle-invasive tumors, which more commonly exhibit dysregulated FGFR3 function via overexpression of the wild-type protein. Recently, a third mechanism of aberrant FGFR3 activation in bladder cancer has been identified that involves chromosomal rearrangements of FGFR3 with one of two different fusion partners: TAAC3 (transforming acid coiled coil 3) or BAI1AP2L1 (BAI1-associated protein 2-like 1). Both FGFR3-TAAC3 and FGFR3-BAI1AP2L1 translocations generate constitutively activated and oncogenic FGFR3 kinase protein products, and cellular dependence on these drivers confers sensitivity to selective FGFR inhibition.

In addition, dysregulation of FGFR3 has been associated with other hematopoietic malignancies. For example, a chromosomal translocation t(4;12) (p16;p13) was reported in one case of human peripheral T-cell lymphoma. The translocation results in expression of a fusion TEL-FGFR3 tyrosine kinase with the N-terminal pointed (PNT) domain of a transcription factor TEL (ETV6) fused to the C-terminal intracellular tyrosine kinase domain of FGFR3. The TEL PNT domain mediates self-association of the fusion protein, and results in constitutive activation of TEL-FGFR3, as has been reported for other TEL-tyrosine kinase fusions such as TEL-PDGFR.

The term “Fibroblast growth factor receptor 3 inhibitor” or “FGFR3 inhibitor” described herein includes any compounds that disrupt the function of fibroblast growth factor receptor 3 production within a cell. FGFR3 inhibitors include BGJ398 (CAS No. CAS 872511-34-7), and AZD4547 (CAS No. CAS 1035270-39-3).

UDP-glucuronosyltransferases (UGTs) are major phase II drug metabolism enzymes in humans. UGTs catalyze the glucuronidation of numerous endogenous compounds such as bilirubin, bile acids, thyroid hormone, and steroid hormones as well as substantial exogenous substrates including therapeutic drugs, carcinogens, and environmental pollutants. Currently, 19 UGT proteins have been identified in humans, and they are divided into three subfamilies, UGT1A, UGT2A, and UGT2B, based on evolutionary divergence and homology. The human UGT1A gene cluster located on chromosome 2q37 contains multiple unique first exons for each UGT1A and common exons 2 to 5, encoding nine kinds of the functional UGT1A subfamily. The UGT2A and UGT2B genes are located on chromosome 4q13, encoding three and seven functional proteins, respectively. The UGT2A1 and UGT2A2 are formed by differential splicing of variable first exons and common exons 2 to 6, likely the UGT1A gene. Meanwhile, UGT2A3 and each UGT2B are encoded by individual genes. It is known that UGT1A and UGT2B play important roles in the glucuronidation of a variety of endogenous and exogenous compounds. The liver plays a central role in metabolism, including glucuronidation. Additionally, extrahepatic tissues such as the gastrointestinal tract and kidney also have a role in metabolism. The distribution of UGT expression in human tissues has been studied mainly in the liver and gastrointestinal tract. Human tissue-derived cell lines are used as a tool for in vitro drug metabolism studies or induction studies. Hepatoma HepG2 cells and adenocarcinoma Caco-2 cells are frequently used, and the expression of selected UGT isoforms in these cell lines has been reported.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer. Examples of Hsp90 client proteins that have been implicated in the progression of cancer are described below.

Hsp90 has been shown by mutational analysis to be necessary for the survival of normal eukaryotic cells. However, Hsp90 is over expressed in many tumor types indicating that it may play a significant role in the survival of cancer cells, and that cancer cells may be more sensitive to inhibition of Hsp90 than normal cells. For example, cancer cells typically have a large number of mutated and overexpressed oncoproteins that are dependent on Hsp90 for folding. In addition, because the environment of a tumor is typically hostile due to hypoxia, nutrient deprivation, acidosis, etc., tumor cells may be especially dependent on Hsp90 for survival. Moreover, inhibition of Hsp90 causes the simultaneous inhibition of a number of oncoproteins, hormone receptors and transcription factors, thus making it an attractive target for an anti-cancer agent. In fact, benzoquinone ansamycins, a family of natural products that inhibit Hsp90, have shown evidence of therapeutic activity in clinical trials.

Although initially promising, first generation Hsp90 inhibitors, the benzoquinone ansamycins, and their derivatives, suffer from some limitations. For example, they have low oral bioavailability and their limited solubility makes them difficult to formulate. In addition, they are metabolized by polymorphic cytochrome P450 CYP3A4 and are a substrate for the P-glycoprotein export pump involved in the development of multidrug resistance. Additionally, the ansamycin class of Hsp90 inhibitors has shown serious toxicity problems. Therefore, a need exists for new therapeutics that improve the prognosis of cancer patients and that reduce or overcome the limitations of currently used anti-cancer agents. Hsp90 inhibitors used herein are resorcinol based agents such as ganetespib (CAS No. 888216-25-9 and NVP-AUY-922 (CAS No. 747412-49-3, alternatively AUY-922).

As used herein, a “subject with a mutation” in FGFR3 gene associated cancer, or a “subject with a cancer with a mutation” in FGFR3, and the like, are understood as a subject having cancer, wherein the tumor has at least one alteration (e.g., 1, 2, 3, 4, 5, 6, 7, 8, 9, 10, or more) in the indicated gene from the wild-type sequence in the gene and/or transcriptional, translational, and/or splicing control regions of the gene that result in the cell becoming cancerous, e.g., developing characteristics such as uncontrolled proliferation, immortality, metastatic potential, rapid growth and proliferation rate, decreased cell death/apoptosis, and certain characteristic morphological features. Mutations include, for example, insertions, deletions, truncations, point mutations, and translocations. Mutations within a gene product can result in constituent activation of the gene product. Mutations that include alterations in transcriptional, translational, or splicing control regions can result in aberrant expression, typically over-expression, of a wild-type gene product. It is understood that not all gene mutations, even in oncogenes, result in a cell becoming cancerous. Mutations that result in oncogenesis are well known in the art. Methods to test mutations for oncogenic activity are well known in the art.

A mutation can be detected using any of a number of known methods in the art. The specific method to detect the mutation will depend, for example, on the type of mutation to be detected. For example, alterations in nucleic acid sequences can be easily detected using polymerase chain reaction and fluorescence in situ hybridization methods (FISH). Protein expression levels can be detected, for example, using immunohistochemistry. An aberrant expression level of a wild-type protein can be used as a surrogate for detection of a mutation in a transcriptional, translational, and/or splicing control regions of the gene without direct detection of the specific genetic change in the nucleic acid in the subject sample. The specific method of detection of the mutation is not a limitation of the invention. Methods to compare protein expression levels to appropriate controls are well known in the art.

Mutations or protein expression levels are preferably detected in a subject sample from the cancer tissue or tumor tissue, e.g., cells, extracellular matrix, and other naturally occurring components associated with the tumor. The mutation or expression level can be detected in a biopsy sample or in a surgical sample after resection of the tumor. The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In an embodiment, the sample is removed from the subject. In a particular embodiment, the sample is urine or serum. In an embodiment, the sample comprises cells. In another embodiment, the sample does not comprise cells. In certain embodiments, the sample can be the portion of the subject that is imaged. Samples are typically removed from the subject prior to analysis; however, tumor samples can be analyzed in the subject, for example, using imaging or other detection methods.

As used herein, the terms “identify” or “select” refer to a choice in preference to another. In other words, to identify a subject or select a subject is to perform the active step of picking out that particular subject from a group and confirming the identity of the subject by name or other distinguishing feature. With respect to the present disclosure, it is understood that identifying a subject or selecting a subject as having one or more mutations in one or more genes of interest, having a wild-type gene, or having a change in the expression level of a protein, and can include any of a number of acts including, but not limited to, performing a test and observing a result that is indicative of a subject having a specific mutation; reviewing a test result of a subject and identifying the subject as having a specific mutation; reviewing documentation on a subject stating that the subject has a specific mutation and identifying the subject as the one discussed in the documentation by confirming the identity of the subject e.g., by an identification card, hospital bracelet, asking the subject for his/her name and/or other personal information to confirm the subjects identity.

As already indicated, the methods and procedures for the detections and/or identifications of FGFR3 over-expressions and/or mutations are known in the literature and can be easily carried out by a skilled person. See, e.g., Chen et al, Oncogene, 2005, 24, 8259-8267; Tomlinson et al, J. Pathol, 2007, 213, 91-98; Williams et al, Human Molecular Genetics, 2013, 22, 795-803; Billerey et al, American Journal of Pathology, 2001, 158, 1955-1959; Nakamura et al, Drug Metabolism and Disposition, 2008, 36, 1461-1464; and Tang et al, Human Molecular Genetics, 2012, 21, 1918-1930; and the references cited in the above identified references. Thresholds of increased expression that constitute an increased level of UGT1A or UGT1A9 expression are easily identifiable in the art.

The “normal” level of expression of UGT1A or UGT1A9 or UGT1A10 is the level of expression of UGT1A or UGT1A9 or UGT1A10 in cells of a subject or patient not afflicted with cancer. In one embodiment, a “normal” level of expression refers to the level of expression of UGT1A or UGT1A9 under normal conditions.

An “over-expression” or “high level of expression” of UGT1A or UGT1A9 or UGT1A10 refers to an expression level in a test sample that is greater than the standard error of the assay employed to assess expression, and is preferably at least 1.1, 1.2, 1.3, 1.4, 1.5, 0.16, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the expression level of UGT1A or UGT1A9 in a control sample (e.g., sample from a healthy subject not having the marker associated disease, i.e., cancer). In one embodiment, expression of UGT1A or UGT1A9 or UGT1A10 is compared to an average expression level of UGT1A or UGT1A9 or UGT1A10 in several control samples.

A “low level of expression” or “under-expression” of UGT1A or UGT1A9 or UGT1A10 refers to an expression level in a test sample that is less than at least 0.9, 0.8, 0.7, 0.6, 0.5, 0.4, 0.3, 0.2, or 0.1 times the expression level of UGT1A or UGT1A9 or UGT1A10 in a control sample (e.g., sample from a healthy subjects not having UGT1A or UGT1A9 or UGT1A10 associated disease, i.e., cancer). In one embodiment, expression of UGT1A or UGT1A9 or UGT1A10 is compared to an average expression level of UGT1A or UGT1A9 or UGT1A10 in several control samples. In one embodiment, a “low level expression” of UGT1A or UGT1A9 or UGT1A10 may also mean that the level of UGT1A or UGT1A9 or UGT1A10 may not be detectable by the traditional/standard detection methods. In one embodiment, a “low level expression” of UGT1A or UGT1A9 or UGT1A10 may also mean that the level of UGT1A or UGT1A9 or UGT1A10 may just be trace amount and cannot be easily quantifiable, but readily identifiable by a skilled person in the art.

As used herein, “detecting”, “detection” and the like are understood that an assay performed for identification of a specific analyte in a sample, e.g., a gene or gene product with a mutation, or the expression level of a gene or gene product in a sample, typically as compared to an appropriate control cell or tissue. The specific method of detection used is not a limitation of the invention. The detection method will typically include comparison to an appropriate control sample.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level of signal detected or protein expression in a control sample that consists of a group of measurements may be determined, e.g., based on any appropriate statistical measure, such as, for example, measures of central tendency including average, median, or modal values.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix.

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which said therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

Other anti-proliferative or anti-cancer therapies may be combined with the compounds described herein to treat proliferative diseases and cancer. Other therapies or anti-cancer agents that may be used in combination with the inventive anti-cancer agents described herein include surgery, radiotherapy (including, but not limited to, gamma-radiation, neutron beam radiotherapy, electron beam radiotherapy, proton therapy, brachytherapy, and systemic radioactive isotopes), endocrine therapy, biologic response modifiers (including, but not limited to, interferons, interleukins, and tumor necrosis factor (TNF)), hyperthermia and cryotherapy, agents to attenuate any adverse effects (e.g., antiemetics), and other approved chemotherapeutic drugs.

The dosages of other anti-cancer agents, which have been or are currently being used to prevent, treat, manage, or ameliorate disorders, such cancer, or one or more symptoms thereof can be used in the combination therapies of the invention. Preferably, dosages lower than those which have been or are currently being used to prevent, treat, manage, or ameliorate cancer, or one or more symptoms thereof, are used in the combination therapies of the invention. The recommended dosages of agents currently used for the prevention, treatment, management, or amelioration of cancer, or one or more symptoms thereof, can obtained from any reference in the art including, but not limited to, Hardman et al., eds., 1996, Goodman & Gilman's The Pharmacological Basis Of Basis Of Therapeutics 9^{th} Ed, Mc-Graw-Hill, N.Y.; Physician's Desk Reference (PDR) 57^{th} Ed., 2003, Medical Economics Co., Inc., Montvale, N.J.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

Some of the disclosed pharmaceutical compositions can be particularly effective at treating subjects with proliferative disorders. In one embodiment, the proliferative disorder is cancer. In one embodiment, the pharmaceutical composition is administered to a subject whose cancer has become “drug resistant” or “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. “Drug resistant” tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

A pharmaceutically acceptable carrier may contain inert ingredients which do not unduly inhibit the biological activity of the compound(s). The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

An “effective amount” is that amount sufficient to treat a disease in a subject. A therapeutically effective amount can be administered in one or more administrations.

The term “effective amount” includes an amount of ganetespib which is sufficient to treat the cancer, to reduce or ameliorate the severity, duration, or progression of cancer, to retard or halt the advancement of cancer, to cause the regression of cancer, to delay the recurrence, development, onset, or progression of a symptom associated with cancer, or to enhance or improve the therapeutic effect(s) of another therapy. For example, an effective amount can induce, for example, a complete response, a partial response, or stable disease; as determined, for example, using RESIST criteria.

An “effective amount” of a therapeutic agent produces a desired response. Having a positive response to treatment with a therapeutic agent is understood as having a decrease in at least one sign or symptom of a disease or condition (e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). It is understood that an “effective amount” need not be curative.

An effective amount of ganetespib is understood as an amount of ganetespib to improves outcome relative to an appropriate control group, e.g., an untreated group, a group treated with a combination of therapies not including ganetespib. Methods to select appropriate control groups and to perform comparative analyses are within the ability of those of skill in the art.

The precise amount of compound administered to provide an “effective amount” of ganetespib to the subject will depend on the mode of administration, the type and severity of the cancer and on the characteristics of the subject, such as general health, age, sex, body weight and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th ed., 2003).

The dosage of an individual agent used in combination therapy may be equal to or lower than the dose of an individual therapeutic agent when given independently to treat, manage, or ameliorate a disease or disorder, or one or more symptoms thereof. In one embodiment, the disease or disorder being treated with a combination therapy is a triple-negative breast cancer.

In an embodiment, the amount of ganetespib administered is from about 2 mg/m² to about 500 mg/m², for example, from about 100 mg/m² to about 500 mg/m², from about 125 mg/m² to about 500 mg/m², from about 150 mg/m² to about 500 mg/m² or from about 175 mg/m² to about 500 mg/m². In an embodiment, the amount of ganetespib administered is about 100 mg/m² to about 300 mg/m², from about 125 mg/m² to about 300 mg/m², from about 150 mg/m² to about 300 mg/m² or from about 175 mg/m² to about 300 mg/m². In some embodiments, the amount of ganetespib administered is about 2 mg/m², 4 mg/m², about 7 mg/m², about 10 mg/m², about 14 mg/m², about 19 m g/m², about 23 mg/m², about 25 mg/m², about 33 mg/m², about 35 mg/m², about 40 mg/m², about 48 mg/m², about 49 mg/m², about 50 mg/m², about 65 mg/m², about 75 mg/m², about 86 mg/m², about 100 mg/m², about 110 mg/m², about 114 mg/m², about 120 mg/m², about 144 mg/m², about 150 mg/m², about 173 mg/m², about 180 mg/m², about 200 mg/m², about 216 mg/m² or about 259 mg/m².

In one embodiment, the pharmaceutical composition is formulated to deliver a dose of about 50 mg/m², about 75 mg/m², about 100 mg/m², about 125 mg/m², about 150 mg/m², about 175 mg/m², about 200 mg/m², about 225 mg/m², or about 250 mg/m². Administration of a compound described herein may occur once a week or twice a week. In one embodiment, the pharmaceutical composition is formulated at a dose of about 200 mg/m² and administered once a week.

In one embodiment, the pharmaceutical composition is administered parentally. In one embodiment, the pharmaceutical composition is administered intravenously through an in-dwelling port or through peripheral access. In one embodiment, the pharmaceutical composition is administered through a silicone catheter in an in-dwelling port.

In one embodiment, the pharmaceutical compositions described herein are administered once or twice every week for three out of four weeks, with the fourth week being a “rest week” for the subject being treated. In one embodiment, the pharmaceutical compositions described herein can also be administered once or twice a week for more than three consecutive weeks, with no rest week.

The language “twice-weekly” includes administration of ganetespib two times in about 7 days. For example, the first dose of ganetespib is administered on day 1, and the second dose of ganetespib may be administered on day 2, day 3, day 4, day 5, day 6 or day 7. In some embodiments, the twice-weekly administration occurs on days 1 and 3 or days 1 and 4.

In some embodiments, ganetespib is cyclically administered twice-weekly. For example, ganetespib is administered for a first period of time, followed by a “dose-free” period, then administered for a second period of time. The language “dose-free” includes the period of time in between the first dosing period and the second dosing period in which no ganetespib is administered to the subject. A preferred cycle is administering ganetespib at a dose described above two times during the week for three consecutive weeks followed by one dose-free week. This cycle is then repeated, as described below.

The language “one cycle” includes the first period of time during which ganetespib is administered, followed by a dose-free period of time. The dosing cycle can be repeated and one of skill in the art will be able to determine the appropriate length of time for such a cyclical dosing regimen. In an embodiment, the cycle is repeated at least once. In an embodiment, the cycle is repeated two or more times. In an embodiment, the cycle is repeated 3, 4, 5, 6, 7, 8, 9, 10, 11, 12, 13, 14, 15 or more times, or as many times as medically necessary as determined by one of skill in the art, e.g., as long as the subject exhibits a response with no dose limiting toxicities. In an embodiment, the cycle is repeated until the patient has been determined to be in partial remission (e.g., 50% or greater reduction in the measurable parameters of tumor growth) or complete remission (e.g., absence of cancer). One of skill in the art can determine a patient's remission status using routine methods well known in the art.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

In an embodiment, the method includes treating, managing, or ameliorating cancer, or one or more symptoms thereof, wherein the cancer has a mutation in FGFR3, comprising administering to a subject in need thereof an effective amount of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof.

In an embodiment, the method includes treating a subject with cancer, the method comprising: obtaining a tissue sample from the cancer of the subject with cancer; determining FGFR3 gene expression in said sample; and treating the subject with an effective amount of ganetespib or NVP-AUY922 if the patient has a FGFR3 mutation; and excluding the subject from ganetespib or NVP-AUY922 therapy if the subject does not have a FGFR3 mutation. For example, the subject is treated with an anti-cancer therapy other than ganetespib or NVP-AUY922, if the subject does not have a FGFR3 mutation and/or low level of UGT1A expression, and/or a low level of UGT1A9 and/or UGT1A10 expression. Exemplary cancers include, multiple myeloma, bladder cancer, cervical carcinoma, urothelial carcinoma, colorectal cancer, or solid tumor, or one or more symptoms thereof.

In an embodiment, the method includes treating, managing, or ameliorating cancer, or one or more symptoms thereof, wherein the cancer has a mutation in FGFR3, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof. In an embodiment, the method includes treating, managing, or ameliorating multiple myeloma, bladder cancer, cervical carcinoma, urothelial carcinoma, or solid tumor, or one or more symptoms thereof, comprising administering to a subject in need thereof about 75 m g/m², or about 100 mg/m², or about 125 mg/m², or about 150 mg/m², or about 175 mg/m², or about 200 mg/m², or about 225 mg/m² of ganetespib, or a pharmaceutically acceptable salt thereof.

In an embodiment, the method includes treating, managing, or ameliorating cancer, or one or more symptoms thereof, wherein the cancer has a low level expression of UGT1A, comprising administering to a subject in need thereof an effective amount of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof.

In an embodiment, the method includes treating a subject with cancer, the method comprising: obtaining a tissue sample from the cancer of the subject with cancer; determining UGT1A gene expression in said sample; and treating the subject with an effective amount of ganetespib or NVP-AUY922 if the subject has a low level of UGT1A expression; and excluding the subject from ganetespib or NVP-AUY922 therapy if the subject does not have a low level of UGT1A expression. For example, the subject is treated with an anti-cancer therapy other than ganetespib or NVP-AUY922, if the subject does not have a FGFR3 mutation and/or low level of UGT1A expression, and/or a low level of UGT1A9 and/or UGT1A10 expression. Exemplary cancers include, multiple myeloma, bladder cancer, cervical carcinoma, urothelial carcinoma, colorectal cancer, or solid tumor, or one or more symptoms thereof.

In an embodiment, the method includes treating, managing, or ameliorating cancer, or one or more symptoms thereof, wherein the cancer has a low level expression of UGT1A, comprising administering to a subject in need thereof an effective amount of ganetespib, comprising administering to a subject in need thereof about 75 m g/m², or about 100 mg/m², or about 125 mg/m², or about 150 mg/m², or about 175 mg/m², or about 200 mg/m², or about 225 mg/m² of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 m g/m², or about 175 mg/m², or about 200 mg/m², or about 225 mg/m² of ganetespib, or a pharmaceutically acceptable salt thereof.

In an embodiment, the method includes treating, managing, or ameliorating cancer, or one or more symptoms thereof, wherein the cancer has a low level expression of UGT1A9, comprising administering to a subject in need thereof an effective amount of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof.

In an embodiment, the method includes treating, managing, or ameliorating cancer, or one or more symptoms thereof, wherein the cancer has a low level expression of UGT1A9, comprising administering to a subject in need thereof an effective amount of ganetespib, comprising administering to a subject in need thereof about 75 m g/m², or about 100 mg/m², or about 125 mg/m², or about 150 mg/m², or about 175 mg/m², or about 200 mg/m², or about 225 mg/m² of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A9, comprising administering to a subject in need thereof an effective amount of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A and/or a low level expression of UGT1A9 and/or UGT1A10, comprising administering to a subject in need thereof an effective amount of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof.

In an embodiment, the method includes treating a subject with cancer, the method comprising: obtaining a tissue sample from the cancer of the subject with cancer; determining FGFR3 gene expression, and/or UGT1A gene expression, and/or UTUGT1A9 and/or UGT1A10 gene expression in said sample; and treating the subject with an effective amount of ganetespib or NVP-AUY922 if the subject has a mutation in FGFR and/or a low level of UGT1A expression, and/or a low level of UGT1A9 and/or UGT1A10 expression; and excluding the subject from ganetespib or NVP-AUY922 therapy if the subject does not have a FGFR3 mutation and/or low level of UGT1A expression, and/or a low level of UGT1A9 and/or UGT1A10 expression. For example, the subject is treated with an anti-cancer therapy other than ganetespib or NVP-AUY922, if the subject does not have a FGFR3 mutation and/or low level of UGT1A expression, and/or a low level of UGT1A9 and/or UGT1A10 expression. Exemplary cancers include, multiple myeloma, bladder cancer, cervical carcinoma, urothelial carcinoma, colorectal cancer, or solid tumor, or one or more symptoms thereof.

In an embodiment, the method includes treating, managing, or ameliorating cancer, or one or more symptoms thereof, wherein the cancer has a mutation in FGFR3 and/or with a low level expression of UGT1A, and/or a low level expression of UGT1A9 and/or UGT1A10, comprising administering to a subject in need thereof an effective amount of ganetespib, comprising administering to a subject in need thereof about 75 m g/m², or about 100 mg/m², or about 125 mg/m², or about 150 mg/m², or about 175 mg/m², or about 200 mg/m², or about 225 mg/m² of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 m g/m², or about 125 mg/m², or about 150 mg/m², or about 175 mg/m², or about 200 mg/m², or about 225 mg/m² of ganetespib, or a pharmaceutically acceptable salt thereof.

In an embodiment, the method includes treating, managing, or ameliorating cancer, or one or more symptoms thereof, wherein the cancer has a low level expression of UGT1A and/or a low level expression of UGT1A9 and/or UGT1A10, comprising administering to a subject in need thereof an effective amount of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof.

In an embodiment, the method includes treating, managing, or ameliorating cancer, or one or more symptoms thereof, wherein the cancer has a low level expression of UGT1A, and/or a low level expression of UGT1A9 and/or UGT1A10, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof.

In an embodiment, the method includes treating, managing, or ameliorating cancer, or one or more symptoms thereof, wherein the cancer has a mutation in FGFR3, comprising administering to a subject in need thereof an effective amount of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof.

In an embodiment, the method includes treating, managing, or ameliorating cancer, or one or more symptoms thereof, wherein the cancer has a mutation in FGFR3, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof.

In an embodiment, the method includes treating, managing, or ameliorating cancer, or one or more symptoms thereof, wherein the cancer has a low level expression of UGT1A, comprising administering to a subject in need thereof an effective amount of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof.

In an embodiment, the method includes treating, managing, or ameliorating cancer, or one or more symptoms thereof, wherein the cancer has a low level expression of UGT1A, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof.

In an embodiment, the method includes treating a subject with cancer, the method comprising: obtaining a tissue sample from the cancer of the subject with cancer; determining UGT1A9 and/or UGT1A10 gene expression in said sample; and treating the subject with an effective amount of ganetespib or NVP-AUY922 if the subject has a low level of UGT1A9 and/or UGT1A10 expression; and excluding the subject from ganetespib or NVP-AUY922 therapy if the subject does not have a low level of UGT1A9 and/or UGT1A10. For example, the subject is treated with an anti-cancer therapy other than ganetespib or NVP-AUY922, if the subject does not have a FGFR3 mutation and/or low level of UGT1A expression, and/or a low level of UGT1A9 and/or UGT1A10 expression. Exemplary cancers include, multiple myeloma, bladder cancer, cervical carcinoma, urothelial carcinoma, colorectal cancer, or solid tumor, or one or more symptoms thereof.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A and/or a low level expression of UGT1A9 and/or UGT1A10, comprising administering to a subject in need thereof an effective amount of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof.

In an embodiment, the method includes treating, managing, or ameliorating cancer, or one or more symptoms thereof, wherein the cancer has a mutation in FGFR3 and/or with a low level expression of UGT1A, and/or a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof.

In an embodiment, the method includes treating, managing, or ameliorating cancer, or one or more symptoms thereof, wherein the cancer has a low level expression of UGT1A and/or a low level expression of UGT1A9 and/or UGT1A10, comprising administering to a subject in need thereof an effective amount of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof.

In an embodiment, the method includes treating, managing, or ameliorating cancer, or one or more symptoms thereof, wherein the cancer has a low level expression of UGT1A, and/or a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof.

In an embodiment, the method includes treating, managing, or ameliorating cancer, or one or more symptoms thereof, wherein the cancer has a mutation in FGFR3, comprising administering to a subject in need thereof an effective amount of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3, comprising administering to a subject in need thereof an effective amount of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with BGJ398.

In an embodiment, the method includes treating, managing, or ameliorating cancer, or one or more symptoms thereof, wherein the cancer has a mutation in FGFR3, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 m g/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398.

In an embodiment, the method includes treating, managing, or ameliorating cancer, or one or more symptoms thereof, wherein the cancer has a low level expression of UGT1A, comprising administering to a subject in need thereof an effective amount of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A, comprising administering to a subject in need thereof an effective amount of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with BGJ398.

In an embodiment, the method includes treating, managing, or ameliorating cancer, or one or more symptoms thereof, wherein the cancer has a low level expression of UGT1A, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A, comprising administering to a subject in need thereof an effective amount of ganetespib, comprising administering to a subject in need thereof about 75 mg/m², or about 100 mg/m², or about 125 mg/m², or about 150 mg/m², or about 175 m g/m², or about 200 mg/m², or about 225 mg/m² of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with BGJ398.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A and/or a low level expression of UGT1A9 and/or UGT1A10, comprising administering to a subject in need thereof an effective amount of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A and/or a low level expression of UGT1A9 and/or UGT1A10, comprising administering to a subject in need thereof an effective amount of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A, and/or a low level expression of UGT1A9 and/or UGT1A10, comprising administering to a subject in need thereof an effective amount of ganetespib, comprising administering to a subject in need thereof about 75 m g/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A, and/or a low level expression of UGT1A9 and/or UGT1A10, comprising administering to a subject in need thereof an effective amount of ganetespib, comprising administering to a subject in need thereof about 75 m g/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398.

In an embodiment, the method includes treating, managing, or ameliorating cancer, or one or more symptoms thereof, wherein the cancer has a low level expression of UGT1A and/or a low level expression of UGT1A9 and/or UGT1A10, comprising administering to a subject in need thereof an effective amount of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A and/or a low level expression of UGT1A9 and/or UGT1A10, comprising administering to a subject in need thereof an effective amount of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of ganetespib, or a pharmaceutically acceptable salt thereof, in combination with BGJ398.

In an embodiment, the method includes treating, managing, or ameliorating cancer, or one or more symptoms thereof, wherein the cancer has a low level expression of UGT1A, and/or a low level expression of UGT1A9 and/or UGT1A10, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A, and/or a low level expression of UGT1A9 and/or UGT1A10, comprising administering to a subject in need thereof an effective amount of ganetespib, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with BGJ398.

In an embodiment, the method includes treating, managing, or ameliorating cancer, or one or more symptoms thereof, wherein the cancer has a mutation in FGFR3, comprising administering to a subject in need thereof an effective amount of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3, comprising administering to a subject in need thereof an effective amount of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with BGJ398.

In an embodiment, the method includes treating, managing, or ameliorating cancer, or one or more symptoms thereof, wherein the cancer has a mutation in FGFR3, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398.

In an embodiment, the method includes treating, managing, or ameliorating cancer, or one or more symptoms thereof, wherein the cancer has a low level expression of UGT1A, comprising administering to a subject in need thereof an effective amount of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A, comprising administering to a subject in need thereof an effective amount of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with BGJ398.

In an embodiment, the method includes treating, managing, or ameliorating cancer, or one or more symptoms thereof, wherein the cancer has a low level expression of UGT1A, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with an FGFR3 inhibitor including BGJ398 and AZD4547.

n an embodiment, the method includes treating, managing, or ameliorating cancer, or one or more symptoms thereof, wherein the cancer has a low level expression of UGT1A9 and/or UGT1A10, comprising administering to a subject in need thereof an effective amount of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with BGJ398.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 75 mg/m2, or about 100 mg/m2, or about 125 mg/m2, or about 150 mg/m2, or about 175 mg/m2, or about 200 mg·m2, or about 225 mg/m2 of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A and/or a low level expression of UGT1A9 and/or UGT1A10, comprising administering to a subject in need thereof an effective amount of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A and/or a low level expression of UGT1A9 and/or UGT1A10, comprising administering to a subject in need thereof an effective amount of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with BGJ398.

In an embodiment, the method includes treating, managing, or ameliorating cancer, or one or more symptoms thereof, wherein the cancer has a mutation in FGFR3 and/or with a low level expression of UGT1A, and/or a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a mutation in FGFR3 and/or with a low level expression of UGT1A, and/or a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398.

In an embodiment, the method includes treating, managing, or ameliorating cancer, or one or more symptoms thereof, wherein the cancer has a low level expression of UGT1A and/or a low level expression of UGT1A9 and/or UGT1A10, comprising administering to a subject in need thereof an effective amount of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A and/or a low level expression of UGT1A9 and/or UGT1A10, comprising administering to a subject in need thereof an effective amount of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof an effective amount of NVP-AUY922, or a pharmaceutically acceptable salt thereof, in combination with BGJ398.

In an embodiment, the method includes treating, managing, or ameliorating cancer, or one or more symptoms thereof, wherein the cancer has a low level expression of UGT1A, and/or a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with an FGFR3 inhibitor including BGJ398 and AZD4547.

In an embodiment, the method includes treating, managing, or ameliorating cancer, or one or more symptoms thereof, wherein the cancer has a low level expression of UGT1A, and/or a low level expression of UGT1A9 and/or UGT1A10, comprising administering to a subject in need thereof an effective amount of NVP-AUY922, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398. In an embodiment, the method includes treating, managing, or ameliorating multiple myeloma, bladder cancer, cervical carcinoma, urothelial carcinoma, colorectal cancer, or solid tumor, or one or more symptoms thereof, comprising administering to a subject in need thereof about 0.5 mg/kg, or about 1 mg/kg, or about 2 mg/kg, or about 4 mg/kg, or about 8 mg/kg, or about 10 mg/kg, or about 12 mg/kg, or about 15 mg/kg, or about 18 mg/kg, or about 20 mg/kg, or about 25 mg/kg, or about 30 mg/kg, or about 35 mg/kg, or about 40 mg/kg, or about 45 mg/kg, or about 50 mg/kg, or about 55 mg/kg, or about 60 mg/kg, or about 65 mg/kg, or about 70 mg/kg, or about 75 mg/kg, or about 80 mg/kg, or about 90 mg/kg, or about 100 mg/kg, or about 110 mg/kg, or about 120 mg/kg, or about 130 mg/kg, or about 140 mg/kg, or about 150 mg/kg, or about 160 mg/kg, or about 170 mg/kg, or about 180 mg/kg, about 190 mg/kg, or about 200 mg/kg of NVP-AUY922, or a pharmaceutically acceptable salt thereof, in combination with BGJ398.

In certain embodiments, the methods include administration of ganetespib or a pharmaceutically acceptable salt or a tautomer thereof, at a dose of 2 mg/m² to 260 mg/m², or in any amount falling within that range.

In certain embodiments, the methods include administration of NVP-AUY922 or a pharmaceutically acceptable salt or a tautomer thereof, at a dose of 0.5 mg/kg to 200 mg/kg, or in any amount falling within that range.

In certain embodiments, the invention further includes administering an FGFR3 inhibitor including BGJ398 and AZD4547. In certain embodiments, the invention further includes administering one or more additional anticancer agents. The one or more additional anticancer agents may be BEZ-235, AZD6244, AZD8055, SN-38, gemcitabine, camptothecin, docetaxel, cisplatin, oxaliplatin, crizotinib, paclitaxel, trastuzumab, or pemetrexed.

In certain embodiments, the levels of UGT1A and UGT1A9 and UGT1A10, and mutations are detected in a subject sample, e.g., a tumor sample, and compared to an appropriate control.

The invention provides methods for identifying a subject for treatment with ganetespib or NVP-AUY922 including, providing a subject sample from the subject, determining the level of UGT1A and/or UGT1A9 and/or UGT1A10 in a tumor from the subject, preferably in vitro, and determining, preferably in vitro if the subject has a tumor with a mutation in FGFR3 wherein a low level expression of UGT1A and/or UGT1A9 and/or UGT1A10 in the sample and a mutation in FGFR3, indicates the subject is likely to respond to therapy with ganetespib or NVP-AUY922.

The invention also provides kits to practice the methods of the invention. For example, a kit can include an instruction for administration of ganetespib or NVP-AUY922 to a subject having cancer with a low level expression of UGT1A and/or UGT1A9 and/or UGT1A10 and/or with a mutation in FGFR3. A kit can also include information on measuring the level of the expression of UGT1A and/or UGT1A9 and/or UGT1A10 and/or on determining a mutation in FGFR3.

The invention also provides a pharmaceutical composition comprising ganetespib or a pharmaceutically acceptable salt thereof, an FGFR3 inhibitor and/or a pharmaceutically acceptable carrier. The invention also provides a pharmaceutical composition comprising ganetespib or a pharmaceutically acceptable salt thereof, BGJ398 and/or a pharmaceutically acceptable carrier. The invention also provides a pharmaceutical composition comprising ganetespib or a pharmaceutically acceptable salt thereof, AZD4547 and/or a pharmaceutically acceptable carrier.

The invention also provides a pharmaceutical composition comprising NVP-AUY922 or a pharmaceutically acceptable salt thereof, an FGFR3 inhibitor and/or a pharmaceutically acceptable carrier. The invention also provides a pharmaceutical composition comprising NVP-AUY922 or a pharmaceutically acceptable salt thereof, BGJ398 and/or a pharmaceutically acceptable carrier. The invention also provides a pharmaceutical composition comprising NVP-AUY922 or a pharmaceutically acceptable salt thereof, AZD4547 and/or a pharmaceutically acceptable carrier.

The invention also provides the use of ganetespib or a pharmaceutically acceptable salt thereof for the manufacture of a medicament for the treatment of a subject with a cancer with a mutation in FGFR3. The invention further provides the use of ganetespib or a pharmaceutically acceptable salt thereof for the manufacture of a medicament for the treatment of a subject with a cancer with a mutation in FGFR3 in combination with an FGFR3 inhibitor. The invention further provides the use of ganetespib or a pharmaceutically acceptable salt thereof for the manufacture of a medicament for the treatment of a subject with a cancer with a mutation in FGFR3 in combination with BGJ398. The invention further provides the use of ganetespib or a pharmaceutically acceptable salt thereof for the manufacture of a medicament for the treatment of a subject with a cancer with a mutation in FGFR3 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of ganetespib or a pharmaceutically acceptable salt thereof for the manufacture of a medicament for the treatment of a subject with a cancer with a low level expression of UGT1A. The invention further provides the use of ganetespib or a pharmaceutically acceptable salt thereof for the manufacture of a medicament for the treatment of a subject with a cancer with a low level expression of UGT1A in combination with an FGFR3 inhibitor. The invention further provides the use of ganetespib or a pharmaceutically acceptable salt thereof for the manufacture of a medicament for the treatment of a subject with a cancer with a mutation in FGFR3 in combination with BGJ398. The invention further provides the use of ganetespib or a pharmaceutically acceptable salt thereof for the manufacture of a medicament for the treatment of a subject with a cancer with a mutation in FGFR3 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of ganetespib or a pharmaceutically acceptable salt thereof for the manufacture of a medicament for the treatment of a subject with a cancer with a low level expression of UGT1A9. The invention further provides the use of ganetespib or a pharmaceutically acceptable salt thereof for the manufacture of a medicament for the treatment of a subject with a cancer with a low level expression of UGT1A9 and/or UGT1A10 in combination with an FGFR3 inhibitor. The invention further provides the use of ganetespib or a pharmaceutically acceptable salt thereof for the manufacture of a medicament for the treatment of a subject with a cancer with a mutation in FGFR3 in combination with BGJ398. The invention further provides the use of ganetespib or a pharmaceutically acceptable salt thereof for the manufacture of a medicament for the treatment of a subject with a cancer with a mutation in FGFR3 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of ganetespib or a pharmaceutically acceptable salt thereof for the manufacture of a medicament for the treatment of a subject with a cancer with a mutation in FGFR3 and/or with a low level expression of UGT1A. The invention further provides the use of ganetespib or a pharmaceutically acceptable salt thereof for the manufacture of a medicament for the treatment of a subject with a cancer with a mutation in FGFR3 in combination with an FGFR3 inhibitor. The invention further provides the use of ganetespib or a pharmaceutically acceptable salt thereof for the manufacture of a medicament for the treatment of a subject with a cancer with a mutation in FGFR3 in combination with BGJ398. The invention further provides the use of ganetespib or a pharmaceutically acceptable salt thereof for the manufacture of a medicament for the treatment of a subject with a cancer with a mutation in FGFR3 and/or with a low level expression of UGT1A in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of ganetespib or a pharmaceutically acceptable salt thereof for the manufacture of a medicament for the treatment of a subject with a cancer with a mutation in FGFR3 and/or with a low level expression of UGT1A9 and/or UGT1A10. The invention further provides the use of ganetespib or a pharmaceutically acceptable salt thereof for the manufacture of a medicament for the treatment of a subject with a cancer with a mutation in FGFR3 in combination with an FGFR3 inhibitor. The invention further provides the use of ganetespib or a pharmaceutically acceptable salt thereof for the manufacture of a medicament for the treatment of a subject with a cancer with a mutation in FGFR3 in combination with BGJ398. The invention further provides the use of ganetespib or a pharmaceutically acceptable salt thereof for the manufacture of a medicament for the treatment of a subject with a cancer with a mutation in FGFR3 and/or with a low level expression of UGT1A9 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of ganetespib or a pharmaceutically acceptable salt thereof for the manufacture of a medicament for the treatment of a subject with a cancer with a mutation in FGFR3 and/or with a low level expression of UGT1A and/or with a low level expression of UGT1A9 and/or UGT1A10. The invention further provides the use of ganetespib or a pharmaceutically acceptable salt thereof for the manufacture of a medicament for the treatment of a subject with a cancer with a mutation in FGFR3 in combination with an FGFR3 inhibitor. The invention further provides the use of ganetespib or a pharmaceutically acceptable salt thereof for the manufacture of a medicament for the treatment of a subject with a cancer with a mutation in FGFR3 in combination with BGJ398. The invention further provides the use of ganetespib or a pharmaceutically acceptable salt thereof for the manufacture of a medicament for the treatment of a subject with a cancer with a mutation in FGFR3 and/or with a low level expression of UGT1A and/or with a low level expression of UGT1A9 and/or UGT1A10 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of NVP-AUY922 or a pharmaceutically acceptable salt thereof for the manufacture of a medicament for the treatment of a subject with a cancer with a mutation in FGFR3. The invention further provides the use of NVP-AUY922 or a pharmaceutically acceptable salt thereof for the manufacture of a medicament for the treatment of a subject with a cancer with a mutation in FGFR3 in combination with an FGFR3 inhibitor. The invention further provides the use of NVP-AUY922 or a pharmaceutically acceptable salt thereof for the manufacture of a medicament for the treatment of a subject with a cancer with a mutation in FGFR3 in combination with BGJ398. The invention further provides the use of NVP-AUY922 or a pharmaceutically acceptable salt thereof for the manufacture of a medicament for the treatment of a subject with a cancer with a mutation in FGFR3 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of NVP-AUY922 or a pharmaceutically acceptable salt thereof for the manufacture of a medicament for the treatment of a subject with a cancer with a low level expression of UGT1A. The invention further provides the use of NVP-AUY922 or a pharmaceutically acceptable salt thereof for the manufacture of a medicament for the treatment of a subject with a cancer with a low level expression of UGT1A in combination with an FGFR3 inhibitor. The invention further provides the use of NVP-AUY922 or a pharmaceutically acceptable salt thereof for the manufacture of a medicament for the treatment of a subject with a cancer with a mutation in FGFR3 in combination with BGJ398. The invention further provides the use of NVP-AUY922 or a pharmaceutically acceptable salt thereof for the manufacture of a medicament for the treatment of a subject with a cancer with a mutation in FGFR3 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of NVP-AUY922 or a pharmaceutically acceptable salt thereof for the manufacture of a medicament for the treatment of a subject with a cancer with a low level expression of UGT1A9 and/or UGT1A10. The invention further provides the use of NVP-AUY922 or a pharmaceutically acceptable salt thereof for the manufacture of a medicament for the treatment of a subject with a cancer with a low level expression of UGT1A9 and/or UGT1A10 in combination with an FGFR3 inhibitor. The invention further provides the use of NVP-AUY922 or a pharmaceutically acceptable salt thereof for the manufacture of a medicament for the treatment of a subject with a cancer with a mutation in FGFR3 in combination with BGJ398. The invention further provides the use of NVP-AUY922 or a pharmaceutically acceptable salt thereof for the manufacture of a medicament for the treatment of a subject with a cancer with a mutation in FGFR3 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of NVP-AUY922 or a pharmaceutically acceptable salt thereof for the manufacture of a medicament for the treatment of a subject with a cancer with a mutation in FGFR3 and/or with a low level expression of UGT1A. The invention further provides the use of NVP-AUY922 or a pharmaceutically acceptable salt thereof for the manufacture of a medicament for the treatment of a subject with a cancer with a mutation in FGFR3 in combination with an FGFR3 inhibitor. The invention further provides the use of NVP-AUY922 or a pharmaceutically acceptable salt thereof for the manufacture of a medicament for the treatment of a subject with a cancer with a mutation in FGFR3 in combination with BGJ398. The invention further provides the use of NVP-AUY922 or a pharmaceutically acceptable salt thereof for the manufacture of a medicament for the treatment of a subject with a cancer with a mutation in FGFR3 and/or with a low level expression of UGT1A in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of NVP-AUY922 or a pharmaceutically acceptable salt thereof for the manufacture of a medicament for the treatment of a subject with a cancer with a mutation in FGFR3 and/or with a low level expression of UGT1A9 and/or UGT1A10. The invention further provides the use of NVP-AUY922 or a pharmaceutically acceptable salt thereof for the manufacture of a medicament for the treatment of a subject with a cancer with a mutation in FGFR3 in combination with an FGFR3 inhibitor. The invention further provides the use of NVP-AUY922 or a pharmaceutically acceptable salt thereof for the manufacture of a medicament for the treatment of a subject with a cancer with a mutation in FGFR3 in combination with BGJ398. The invention further provides the use of NVP-AUY922 or a pharmaceutically acceptable salt thereof for the manufacture of a medicament for the treatment of a subject with a cancer with a mutation in FGFR3 and/or with a low level expression of UGT1A9 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

The invention also provides the use of NVP-AUY922 or a pharmaceutically acceptable salt thereof for the manufacture of a medicament for the treatment of a subject with a cancer with a mutation in FGFR3 and/or with a low level expression of UGT1A and/or with a low level expression of UGT1A9 and/or UGT1A10. The invention further provides the use of NVP-AUY922 or a pharmaceutically acceptable salt thereof for the manufacture of a medicament for the treatment of a subject with a cancer with a mutation in FGFR3 in combination with an FGFR3 inhibitor. The invention further provides the use of NVP-AUY922 or a pharmaceutically acceptable salt thereof for the manufacture of a medicament for the treatment of a subject with a cancer with a mutation in FGFR3 in combination with BGJ398. The invention further provides the use of NVP-AUY922 or a pharmaceutically acceptable salt thereof for the manufacture of a medicament for the treatment of a subject with a cancer with a mutation in FGFR3 and/or with a low level expression of UGT1A and/or with a low level expression of UGT1A9 and/or UGT1A10 in further combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed.

Ganetespib and optionally, one or more additional anti-cancer agents, can be administered to a subject by routes known to one of skill in the art. Examples of routes of administration include, but are not limited to, parenteral, e.g., intravenous, intradermal, subcutaneous, oral, intranasal (e.g., inhalation), transdermal, topical, transmucosal, and rectal administration. The agents can be administered by different routes of administration.

Ganetespib and optionally, one or more additional anti-cancer agents, may be formulated with a pharmaceutically acceptable carrier, diluent, or excipient as a pharmaceutical composition. Pharmaceutical compositions and dosage forms of the invention comprise one or more active ingredients in relative amounts and formulated in such a way that a given pharmaceutical composition or dosage form can be used to treat cancer. Administration in combination does not require co-formulation.

A pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In some embodiments, ganetespib is formulated at a concentration of 8 mg/mL in 90% v/v PEG 300 and 10% v/v Polysorbate 80 for intravenous administration.

In an embodiment, the invention also provides a pharmaceutical composition further comprises one or more other therapies (e.g., one or more therapeutic agents that are currently being used, have been used, are known to be useful or in development for use in the treatment or amelioration of a proliferative disorder, such as cancer, or one or more symptoms associated with said proliferative disorder). In one embodiment, the pharmaceutical composition further comprises an additional pharmaceutically acceptable co-solvent. In one embodiment, the pharmaceutical composition described herein is administered to a subject in addition to a second pharmaceutical composition containing one or more additional therapeutic agents.

In one embodiment, the two pharmaceutical compositions containing the two different therapies can be administered sequentially or concurrently. In one embodiment, the administration of a second pharmaceutical composition in addition to the pharmaceutical composition described herein can reduce the effective dosage of one or more of the therapies. In one embodiment, the two pharmaceutical compositions may be administered to a subject by the same or different routes of administration.

The pharmaceutical composition of the second therapeutic agent can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

The invention is illustrated by the following examples, which are not intended to be limiting in any way.

### EXAMPLES

### Materials and Methods

### Cell Lines, Antibodies, and Reagents

The RT4 and SW780 cell lines were obtained from the American Type Culture Collection (ATCC). Each was maintained according to suppliers' instructions, authenticated by routine company DNA typing, and used within six months of receipt. RT112 cells were obtained from Sigma-Aldrich. All primary antibodies were purchased from Cell Signaling Technology with the exception of the anti-UGT1A9 (Sigma Aldrich), FGFR3 (B9) and glyceraldehyde-3-phosphate dehydrogenase (GAPDH) antibodies (Santa Cruz Biotechnology, Inc.). Ganetespib [3-(2,4-dihydroxy-5-isopropylphenyl)-4-(1-methyl-1H-1,2,4-triazol-5(4H)-one] was synthesized by Synta Pharmaceuticals Corp. 17-AAG and 17-DMAG were purchased from LC Laboratories and BGJ398 and AUY922 from Selleck Chemicals.

### Cell Culture, Transfections and Treatments for Colorectal Cancer Cells

Eleven cell lines derived from human colorectal cancers (CRC) were cultured in RPMI (Invitrogen, Darmstadt, Germany) supplemented with 10% FCS (ThermoScientific, Waltham Mass., USA), 2 mM L-glutamine and Penicillin-Streptomycin (Invitrogen). Cell-line cross-contaminatino was excluded using short tandem repeat profiling. Specific knockdown of target genes was performed using pre-designed Silencer Select siRNAs (s231075 and s231076, designated s75 and s76) or control siRNAs (scrambled, all from Ambion, Hamburg, Germany) with a final concentration of 5 nM. Overexpression was performed with pCMV-SPORT6 vector constructs. For both up- and downregulation, Lipofectamine 2000 (Invitrogen) was applied in a forward transfection protocol. Ganetespib (Synta Pharma, Lexington Mass., USA) and 17AAG (Calbiochem, Darmstadt, Germany) were dissolved in DMSO (50 mM and 5 mM stocks, respectively) and diluted to the indicated concentrations with culture medium.

### Viability Assays

Cellular viability was assessed using the CellTiter-Glo Luminescent Cell Viability Assay (Promega) according to manufacturer protocols. Bladder cancer cell lines were seeded into 96-well plates based on optimal growth rates determined empirically for each line. Twenty-four hours after plating, cells were dosed with graded concentrations of ganetespib for 72 hours. CellTiter-Glo was added (50% v/v) to the cells, and the plates incubated for 10 minutes prior to luminescent detection in a SpectraMax Plus 384 microplate reader (Molecular Devices). Data were normalized to percent of control and IC₅₀ values used to determine the sensitivity of each line. For the comparative analyses with ganetespib and BGJ398, RT112 cells were treated with graded concentrations of each compound for 72 hours and cell viability measured as above. For the evaluation of apoptotic induction, caspase activity was assessed using the Caspase-Glo 3/7 assay (Promega) according to manufacturer protocols. Twenty-four hours after plating, RT112 cells were treated with graded concentrations of ganetespib for 24 hours. Caspase-Glo was added to cells (50% v/v) and the plates incubated for 1 hour prior to luminescent detection.

### Western Blotting

Following in vitro assays, tumor cells were disrupted in lysis buffer on ice for 10 minutes. Lysates were clarified by centrifugation and equal amounts of proteins resolved by SDS-PAGE before transfer to nitrocellulose membranes (Bio-Rad). Membranes were blocked with StartingBlock T20 blocking buffer (Thermo Scientific) and immunoblotted with the indicated antibodies. Antibody-antigen complexes were visualized using an Odyssey system (LI-COR).

### Reverse Phase Protein Array

RT112 cells were treated with dimethyl sulfoxide (DMSO, control) or 100 nM ganetespib for 24 hours. Lysates were prepared as recommended by MD Anderson Cancer Center (Houston, Tex.), arrayed on nitrocellulose-coated FAST slides (Whatman) and probed for a standard list of antibodies.

### In Vivo Xenograft Tumor Models

CD-1 nude mice (Charles River Laboratories, Wilmington, Mass.) at 7-12 weeks of age were maintained in a pathogen-free environment and all in vivo procedures were approved by the Synta Pharmaceuticals Corp. Institutional Animal Care and Use Committee. RT112 cells (5×10⁶) were subcutaneously implanted into female mice and animals bearing established tumors (˜150 mm³) were randomized into treatment groups of 8. Mice were i.v. dosed with ganetespib (150 mg/kg) once weekly or p.o. dosed with BGJ398 (10 mg/kg) daily, either alone or in combination, for 3 weeks. Tumor growth inhibition was monitored by tumor volume measurements twice weekly. As a measurement of in vivo efficacy, the % T/C value was determined from the change in average tumor volumes of each treated group relative to the vehicle treated animals. Statistical significance was determined using two-way ANOVA followed by Bonferroni post tests.

### Pharmacodynamics

Nude mice bearing established RT112 xenografts were randomized into groups of 3 and administered a single bolus injection of ganetespib (150 mg/kg) or vehicle. At 24 and 72 hours post treatment, tumors were resected and homogenized in lysis buffer. Expression changes in cell signaling pathway components were interrogated using Pathscan RTK Signaling and Pathscan Intracellular Signaling Array Kits (Cell Signaling Technology) according to manufacturer protocols. Fluorescence readouts were measured using an Odyssey system (LI-COR) and average changes in signaling protein expression were calculated for each cohort.

### Bioanalysis

In the first set of experiments, RT112 and SW780 cells were treated with graded concentrations of ganetespib or 17-DMAG (10-1000 nM) for 1 and 24 hours. Cell lysates were prepared for measurement of intracellular concentrations of the respective HSP90 inhibitors. RT112 and SW780 cells were also treated with 1 μM ganetespib for 15 minutes, 1, 4, 8 and 24 hours. At each time point cell lysates were prepared and media was collected for analysis of intracellular and secreted concentrations of ganetespib and its glucuronidated metabolites. Bioanalysis was performed on equal protein concentrations from cell lysates or equal volumes of media. All samples were ultimately extracted by protein precipitation and analyzed by LC-MS/MS using an Agilent 1100 HPLC interfaced to an API 4000 tandem mass spectrometer (Applied Biosystems). A Phenomenex Kinetex 2.6 μm C18 (30×2.1 mm) column was used with a run time of 3.5 min per sample.

### Gene Expression Analysis

Total RNA was extracted from untreated SW780 and RT112 cells using the RNeasy Kit (Qiagen Inc.). cDNA (1 μg) was synthesized using an RT2 First Strand Synthesis kit (Qiagen) and analyzed using a Human Drug Metabolism: Phase II Enzymes PCR Array and the RT2 SYBR-Green/Rox PCR Master Mix (SABiosciences/Qiagen). Data were normalized using multiple housekeeping genes and analyzed by comparing 2-ACt of the normalized sample. PCR was performed on a Bio-Rad iCycler iQ5 (Bio-Rad).

Statistical Analysis of Gene Expression Patterns and their Correlation with Drug Sensitivities for Colorectal Cancer Cells

mRNA expression levels were analyzed using log 2 transformation and quantile normalization. Except for control spots, all 43,376 features were used without any a priori filtering. In order to determine significant differences of expression levels between the pooled groups of ganetespib-sensitive and ganetespib-resistant cell lines, a moderated Student's t test was computed on a gene-by-gene basis. An empiric Bayes estimator was applied to compute the linear models for thousands of genes in parallel and assess their significance.

In order to not exceed a false-discovery rate (FDR) of 5%, the p-values were adjusted for multiple testing using the Benjamini-Hochberg method. All analyses were performed using the free statistical software R (version 2.15.2; available from: www.r-project.org). Linear models were computed using the limma package

### Gene Expression Analysis by RT-PCR for Colorectal Cancer Cells

Total RNA from cells was isolated using Trizol reagent (Invitrogen) according to manufacturer's guidelines. Equal amounts of RNA were reverse-transcribed using M-MuLV Reverse Transcriptase (NEB) and real-time PCR analysis was performed using qPCR Master-Mix (75 mM Tris-HCl, pH 8.8, 20 mM (NH₄)₂SO₄, 0.01% Tween-20, 3 mM MgCl₂, SYBR Green 1:80,000, 0.2 mM dNTPs, 20 U/ml Taq-polymerase, 0.25% TritonX-100, 0.3M Trehalose and 0.3 mM primers). Primers for total UGT1A: 5′-ATCTGCTTGGTCACCCGATG-3′ and 5′-TCCATGCGCTTTGCATTGTC-3′; for UGT1A1: 5′-GCCATTCCAAAGGGAGGATGTG-3′ and 5′-TGGGAACAGCCAGACAAAAGC-3′; for cluster UGT1A3-5: 5′-CATAATGAGGCCCTGATCAGGC-3′ and 5′-AATCGACAGGTACTTAGCCAG-3′; for UGT1A6: 3′-GCTGGTGGTCCCTCAGGAC-5′ and 5′-CAGCTCTTCTTGGTCATACGGC-3′; for cluster UGT1A7-10: 5′-CACAGTGCCCTGCTCCTC-3′ and 5′-GTTTGGAGAATTTCAGAGGCTATTTC-3′; for normalizer HPRT1: 5′-ATGCTGAGGATTTGGAAAGG-3′ and 5′-TCATCACATCTCGAGCAAGAC-3′. Primers were used in a two-step protocol (2 min at 95° C. pre-heating; 40 cycles at 95° C. for 15 s followed by 58° C. for 1 min).

### Immunoblot Analysis and Antibodies for Colorectal Cancer Cells

Whole cell lysates were made with RIPA buffer (1% TritonX-100, 1% Desoxycholat, 0.1% SDS, 150 mM NaCl, 10 mM EDTA, 20 mM Tris-HCl, pH 7.5, and complete protease inhibitor mix) and homogenized by sonication. After protein determination by BCA protein assay (Pierce, Bonn, Germany), equal amounts of total protein were separated by SDS gel electrophoresis, transferred onto nitrocellulose membrane (Millipore, Darmstadt, Germany), blocked and probed with the following antibodies (all in 5% milk in Tris-buffered saline solution containing 0.1% Tween-20): Wee1 (cs4936), AKT (cs9272; both Cell Signaling, Frankfurt, Germany), GAPDH (ab8245; abcam, Cambridge, UK), UGT1A (sc-271268), phos-ERK (sc-7383), ERK (sc-94; Santa Cruz, Heidelberg, Germany). Antibodies were detected with peroxidase-coupled secondary antibodies (Jackson, Newmarket, UK).

### Proliferation Assay for Colorectal Cancer Cells

For cell proliferation analysis, cells were seeded at 105 cells/well in 12-well plates 24 hours prior to treatment. Cells were then treated with inhibitors with continuous change of inhibitor-containing medium and confluence measurement every 24 hours. Over the course of the 4-day treatment, cell confluence was measured by brightfield microscopy using a Celigo Adherent Cell Cytometer (Brooks, Chelmsford Mass., USA). Confluence was calculated with the Celigo software program.

### Viability Assay for Colorectal Cancer Cells

24 hours after transfection, 104 cells were seeded in triplicate in 96-well plates one day before treatment. Cells were treated with inhibitors and drugs for 48 hours and subjected to the CellTiterGlo Luminescent Cell Viability assay (Promega, Madison Wis., USA).

### Bioanalysis for Colorectal Cancer Cells

Colon cancer cells were treated with 1 μM ganetespib for 5, 15, 30, 60 or 480 minutes. At each time point, media was collected and cell lysates were generated for subsequent bioanalysis of secreted and intracellular concentrations of ganetespib and its glucuronides. Equal protein concentrations from cell lysates or equal volumes of media were used for the bioanalysis. Samples were extracted by protein precipitation and analyzed by LC-MS/MS using an Agilent 1100 HPLC interfaced to an API 4000 tandem mass spectrometer (Applied Biosystems). A Phenomenex Kinetex 2.6 μm C18 (30×2.1 mm) column was used with a run time of 3.5 min per sample.

### Example 1

### Ganetespib Displays Potent Cytotoxic Activity in FGFR3-Driven Bladder Cancer Lines

Sensitivity to targeted HSP90 inhibition by ganetespib was evaluated using a panel of 20 bladder cancer cell lines of diverse genetic FGFR3 backgrounds (Table 1 below). In the majority of lines examined ganetespib reduced cellular viability with low nanomolar potency, including all that expressed activating point mutations. Further, ganetespib treatment could overcome the FGFR inhibitor-resistant phenotype displayed by mutant FGFR3-expressing 97-7 and MGH-U3 cells. These lines harbor FGFR3Y249C and FGFR3Y375C mutations, respectively, which confer resistance to the pan-FGFR inhibitor BGJ398 yet both remained susceptible to thecytotoxic effects of ganetespib exposure.

TABLE 1
In vitro cytotoxicity values of ganetespib in bladder cancer cell lines.

| | Ganetespib IC₅₀ | |
|---|---|---|
| Cell line | (nM) | FGFR3 status |
| DHS1 | 6 | WT |
| SW-1710 | 6 | WT |
| T24 | 7 | WT |
| RT112 | 9 | FGFR3-TACC3 |
| 639-V | 10 | R248C |
| SCaBER | 10 | WT |
| BFTC | 17 | WT |
| J82 | 18 | K652E |
| HT-1376 | 21 | WT |
| 647-V | 27 | WT |
| UM-UC3 | 33 | WT |
| LB831-BLC | 34 | WT |
| KU-19-19 | 36 | WT |
| 97-7 | 38 | S249C |
| 5637 | 44 | WT |
| HT-1197 | 53 | WT |
| MGH-U3 | 53 | Y375C |
| TCCSUP | 142 | WT |
| RT4 | 1733 | FGFR3-TACC3 |
| SW780 | 3451 | FGFR3-BAIAP2L1 |

### Example 2

### Suppression of Multiple Oncogenic Signaling Cascades in FGFR3 Fusion-Driven RT112 Bladder Cancer Cells by Ganetespib

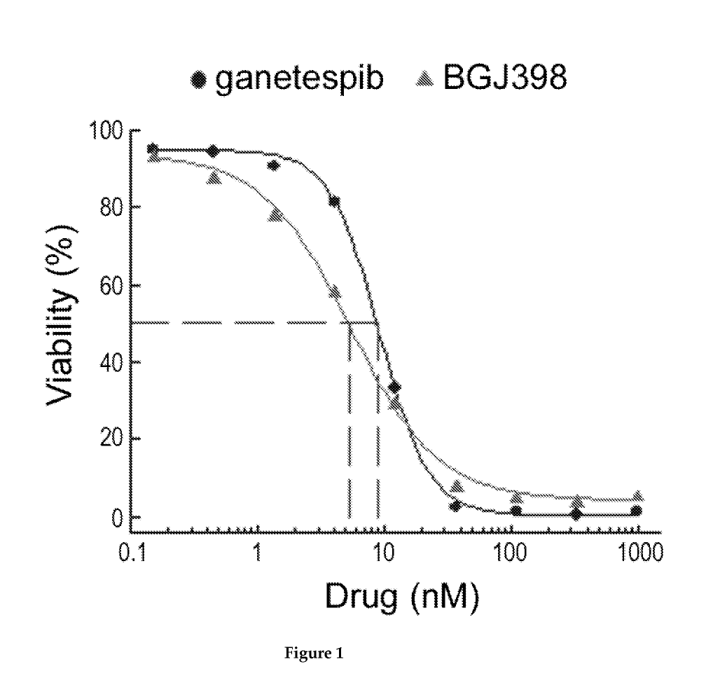

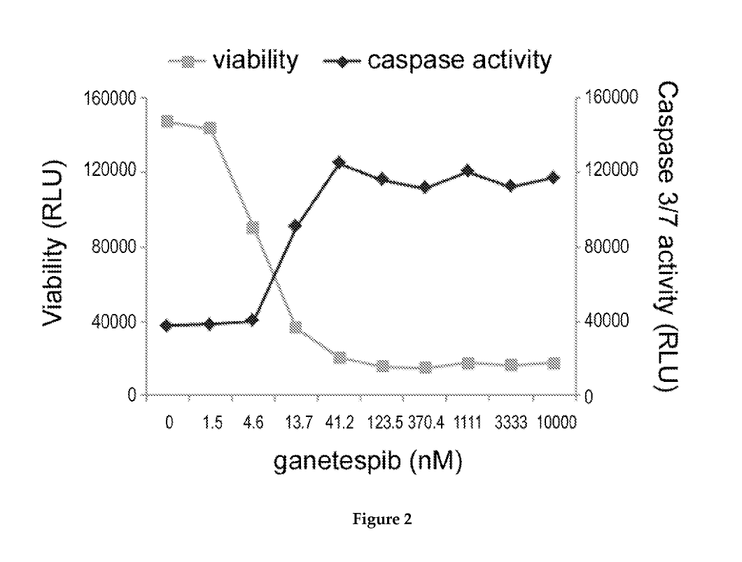

From the initial analysis, RT112 cells were found to be acutely sensitive to ganetespib treatment (IC₅₀ value 9 nM, Table 1). Long considered over-expressers of wild type FGFR3, the recent identification of the FGFR3-TACC3 fusion gene product in these cells accounts for their critical dependence on FGFR3 activity for growth and survival. Moreover, bladder cell lines now known to be FGFR3 fusion-positive, including RT112, have been reported to be sensitive to BGJ398. In this regard, ganetespib was equipotent to selective FGFR inhibition in reducing cell viability in this line (FIG. 1). This loss of viability was concurrent with activation of apoptosis as shown in FIG. 2. RT112 cells were exposed to increasing concentrations of ganetespib and viability measured at 72 hours. This profile was compared to apoptotic induction determined by activated caspase 3/7 levels assessed 24 hours post treatment, which showed that ganetespib-induced cytotoxicity was mediated by an irreversible commitment to apoptosis.

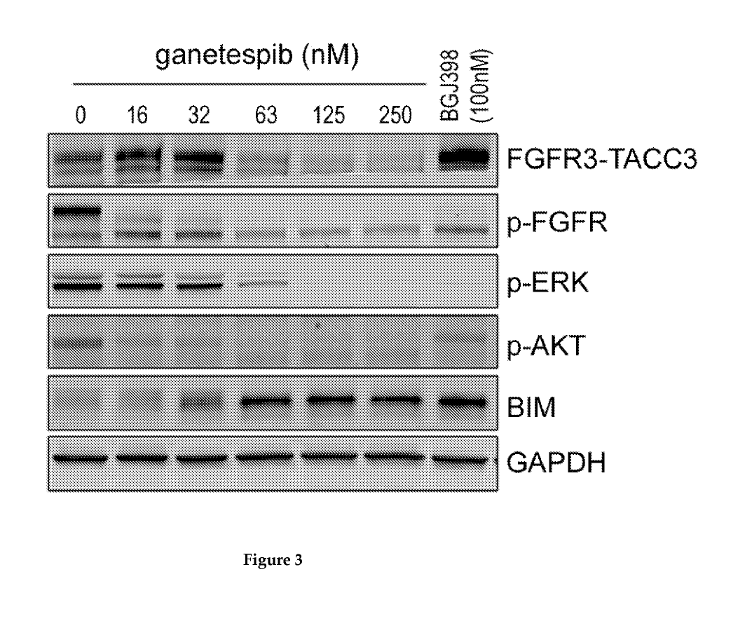

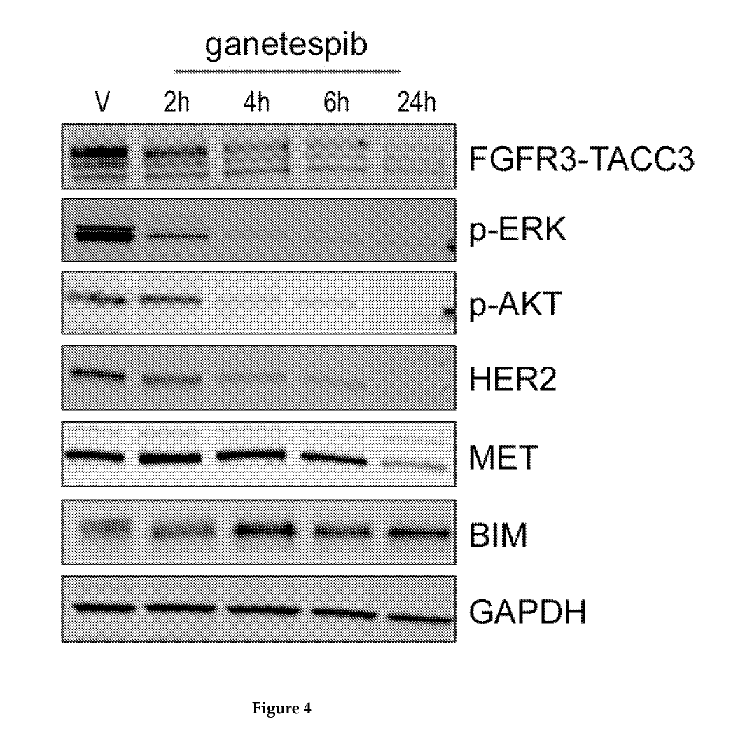

Next, examinations were performed to determine the molecular changes in client and signaling protein pathways associated with FGFR3-TAAC3 in ganetespib-treated RT112 cells (FIG. 3). Ganetespib induced a robust and dose-dependent destabilization of FGFR3-TAAC3 expression, in terms of both total and phosphorylated protein levels, suggesting that the FGFR3 fusion protein was highly responsive to Hsp90 inhibition. Importantly, targeted degradation of this oncogenic driver was accompanied by loss of downstream signaling effector activity (as evidenced by loss of phosphorylated ERK and AKT levels) and induction of BIM, an additional marker of apoptosis (FIG. 3). Consistent with its alternative mode of action as a specific tyrosine kinase inhibitor, an effective dose of BGJ398 (100 nM) did not affect total FGFR3-TAAC3 expression but resulted in a similar abrogation of autophosphorylated FGFR3-TAAC3 activity, disruption of oncogenic pathways, and induction of apoptosis. When the kinetics of client protein loss in response to HSP90 inhibition were examined (FIG. 4), it was found that destabilization of the FGFR3-TAAC3 fusion and the congruent alterations in downstream signaling were relatively rapid, occurring within 4 hours of ganetespib exposure.

A feature of targeted HSP90 blockade is the simultaneous disruption of multiple cellular signaling cascades and processes that are exquisitely dependent on the chaperoning function of the molecule. Therefore, a more extensive reverse phase protein array analysis was performed for ganetespib on its effects in RT112 cells. In addition to the expected downregulation of client receptor tyrosine kinases such as HER2, MET and EGFR, ganetespib treatment also selectively altered the expression of a number of proteins involved in mitogen-activated protein kinase (MAPK), AKT, and mammalian target of rapamycin (mTOR) signaling and cell cycle regulation, along with predicted increases in the apoptotic markers caspase 7 and BIM (See Table 2 below). Thus, the potent and selective disruption of FGFR3-mediated signaling coupled with coordinate effects on additional mitogenic and survival pathways in RT112 cells accounts for the potent cytotoxic activity of ganetespib in this line.

TABLE 2
Fold-changes in protein expression following ganetespib treatment
in RT112 bladder cancer cells using reverse phase protein
array analysis.

| Cellular Target | Protein | Fold Change |
|---|---|---|
| Receptor Tyrosine Kinases | MET (pY1235) | −2.7 |
| | HER2 | −2.2 |
| | HER2 (pY1248) | −2.2 |
| | EGFR (pY1068) | −1.9 |
| | HER3 | −1.6 |
| | EGFR | −1.5 |
| AKT signaling | AKT (pS473) | −2.5 |
| | AKT | −2.4 |
| | GSK3-A/B (pS2/S9) | −1.8 |
| | PDK1 (pS241) | −1.7 |
| | GSK3-A/B | −1.5 |
| | PDK1 | −1.4 |
| MAPK pathway | C-RAF | −1.8 |
| | MAPK (pT202/Y204) | −1.5 |
| | MEK1 (pS217/S221) | −1.4 |
| | Src (pY527) | −1.4 |
| | P90RSK (pT359/S363) | −1.4 |
| Transcription factors | c-Myc | −2.5 |
| | NF-κB p65 (pS536) | −2.3 |
| mTOR pathway | S6 (pS235/S236) | −13.2 |
| | S6 (pS240/S244) | −8.9 |
| | P70S6K (pT389) | −4.7 |
| | 4E-BP1 (pS65) | −2.5 |
| | mTOR (pS2448) | −2.2 |
| | Tuberin | −2.2 |
| | TSC1 | −1.3 |
| | PRAS40 (pT246) | −1.2 |
| Cell cycle regulation | Rb (pS807/811) | −4.6 |
| | Chk1 | −3.2 |
| | Cyclin B1 | −1.9 |
| | CDK1 | −1.4 |
| | p21 | +1.4 |
| | p27 | +1.5 |
| Stress response | HSP70/72 | +3.0 |
| | HSP90 alpha | +1.6 |
| Apoptosis | Caspase 7 (cleaved D198) | +2.5 |
| | BIM | +1.4 |

### Example 3

### Ganetespib in Combination with FGFR3 Tyrosine Kinase Inhibition Confers Superior Antitumor Activity In Vitro and In Vivo Suppression of Multiple Oncogenic Signaling Cascades in FGFR3 Fusion-Driven RT112 Bladder Cancer Cells by Ganetespib

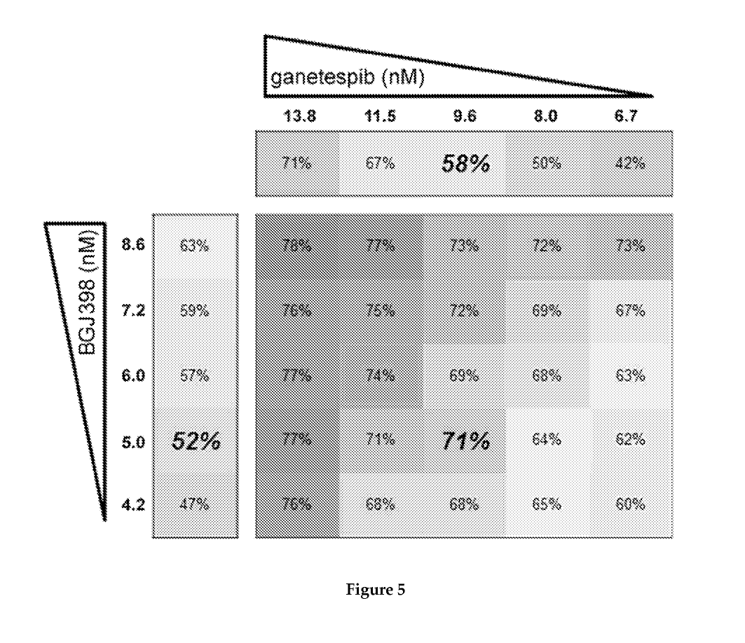

Based on their distinct mechanisms of action on FGFR3-TAAC inhibition, experiments were conducted to determine whether combining ganetespib and BGJ398 would lead to increased activity in FGFR3 fusion-dependent bladder cancer cells. Initial evaluation was assessed of the effects on cellular viability following combination exposure in the RT112 cell line (FIG. 5). The bladder cancer cells were treated with increasing concentrations of ganetespib and/or BGJ398 and viability assessed after 72 hours. The percentages represent the degree of cell death at each individual dose level, which revealed that combinatorial benefit was achieved at all concentrations tested. For example, at the approximate IC₅₀ concentrations for each inhibitor (9.6 nM for ganetespib and 5 nM for BGJ398), combined exposure resulted in 71% cell killing. Overall, all combinations of ganetespib and BGJ398 showed much improved cell killing activity over single-agent treatment alone.

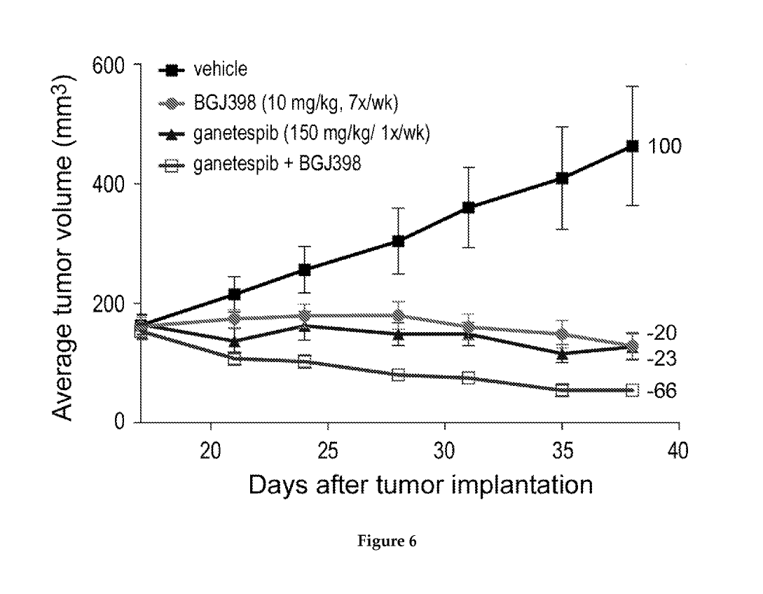

To determine whether these effects on cell viability in vitro would translate to improved efficacy in vivo, RT112 xenograft bearing mice were treated with ganetespib and BGJ398, both as single agents and in combination. It has previously been determined that the maximally tolerated dose of ganetespib on a weekly dosing regimen is 150 mg/kg (17). Weekly administration of ganetespib at this dosing level was comparable to daily dosing of BGJ398 at 10 mg/kg, with each compound inducing a similar degree of tumor regression (T/C values of −23% and −20%, respectively; FIG. 6). Concurrent treatment with both drugs resulted in an enhancement of antitumor activity, causing 66% tumor regression. In addition, combination treatment was well tolerated, with no significant changes in body weights seen after 3 weeks of treatment. Thus, ganetespib and BGJ398, when combined, displayed superior antitumor efficacy compared to monotherapy in RT112 bladder tumor xenografts.

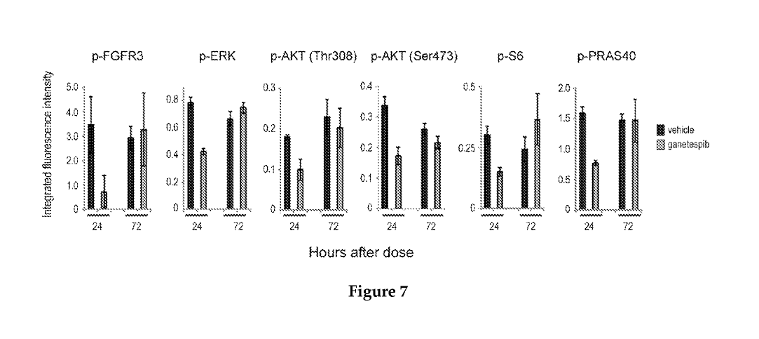

Next, pharmacodynamic analysis was performed in additional mice bearing RT112 xenografts to confirm that the ganetespib-induced tumor response correlated with target modulation in vivo. Animals were treated with a bolus injection of ganetespib at 150 mg/kg and tumors harvested 24 and 72 hours later. Control group mice were administered a single injection of vehicle and tumors excised at the same time points. Expression changes in components of multiple signaling pathways, including receptor tyrosine kinases and their effector proteins, were investigated using multiplexed antibody arrays; average changes for each treatment cohort are presented in FIG. 7. Ganetespib exposure resulted in the deactivation of endogenous FGFR3-TAAC3 activity, as evidenced by the significant repression of p-FGFR3 levels and congruent repression of phosphorylated ERK and AKT by 24 hours. These effects were sustained over time with recovery occurring at 72 hours post treatment. Similar kinetics were observed for signaling intermediates of the mTOR pathway (phosphorylated S6 ribosomal protein and PRAS40), consistent with what was observed following targeted HSP90 inhibition in vitro (Table 2). Overall, these data show that single-dose ganetespib exerts a potent and rapid destabilizing effect on the FGFR3-TAAC3 fusion kinase and its effectors in RT112 xenografts.

### Example 4

### Differential Sensitivity of FGFR3 Fusion-Positive Bladder Cancer Lines to HSP90 Inhibitors

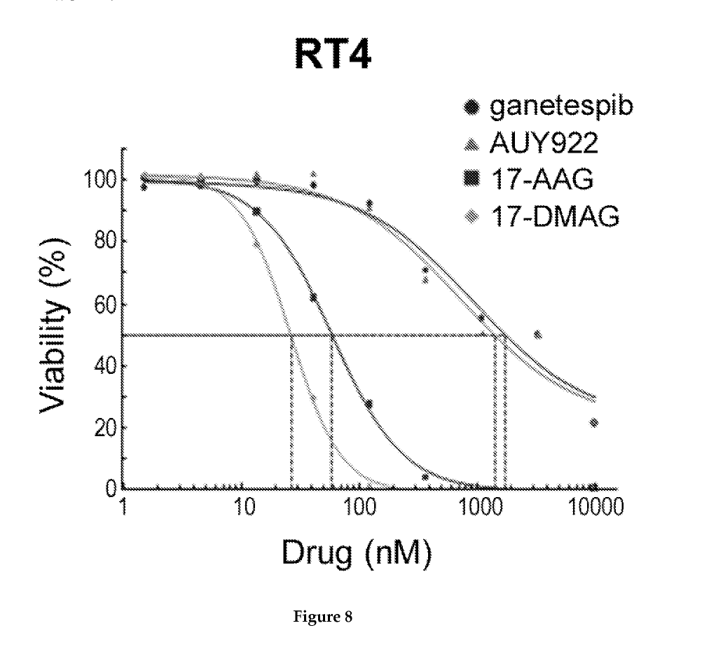

As part of a previous study it was shown that RT112 and RT4 bladder cancer cells, which also express the FGFR3-TACC3 fusion protein, were sensitive to targeted HSP90 inhibition by the first-generation ansamycin compound 17-AAG (17-allylamino-17-demethoxygeldanamycin). However, RT4 cells were largely insensitive to ganetespib exposure (See Table 1). To explore this lack of activity with ganetespib, evaluation was performed of the cytotoxicity of four HSP90 inhibitors (HSP90i) in RT4 and SW780 cells: 17-AAG, the closely related ansamycin analog 17-DMAG (17-dimethylaminoethylamino-17-demethoxygeldanamycin), ganetespib, and another resorcinol-based second-generation inhibitor AUY922 (FIG. 8). In RT4 cells, exposure to an ansamycin-based HSP90i resulted in dose-dependent cytotoxicity and low nanomolar IC₅₀ values. In contrast, ganetespib and AUY922 were minimally effective at reducing cellular viability with IC₅₀ values greater than 1 μM (FIG. 8).

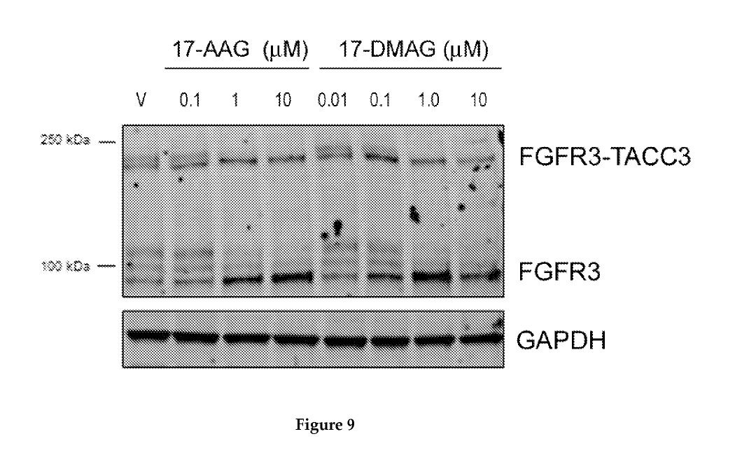

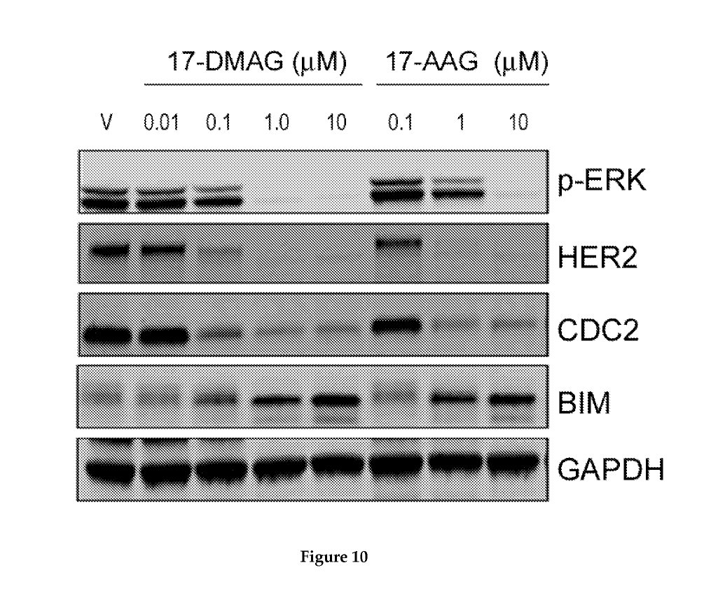

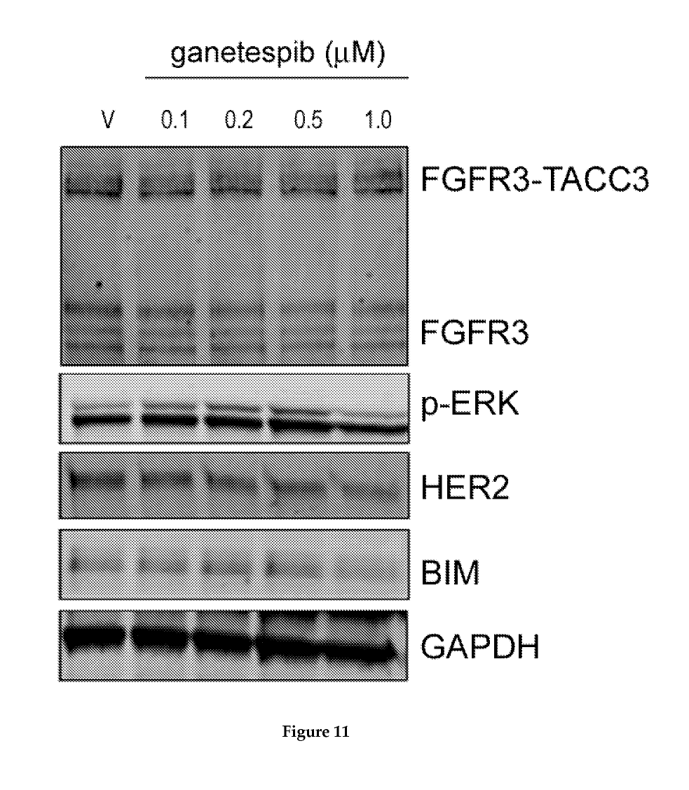

At the molecular level, 17-DMAG and 17-AAG both effectively destabilized FGFR3 and higher weight FGFR3-TAAC3 fusion protein expression in RT4 cells (FIG. 9). This was accompanied by a concomitant loss of downstream ERK activity (FIG. 10). In addition, two other sensitive HSP90 client proteins, HER2 and CDC2, were similarly degraded in a concentration-dependent manner. Consistent with the viability results, a corresponding dose-dependent increase in BIM expression was observed supporting the premise that both of these first-generation HSP90i induced apoptosis in RT4 cells (FIG. 10). As predicted by the sensitivity profile of this line, ganetespib had negligible effects on the expression of any of the same molecular markers (FIG. 11).

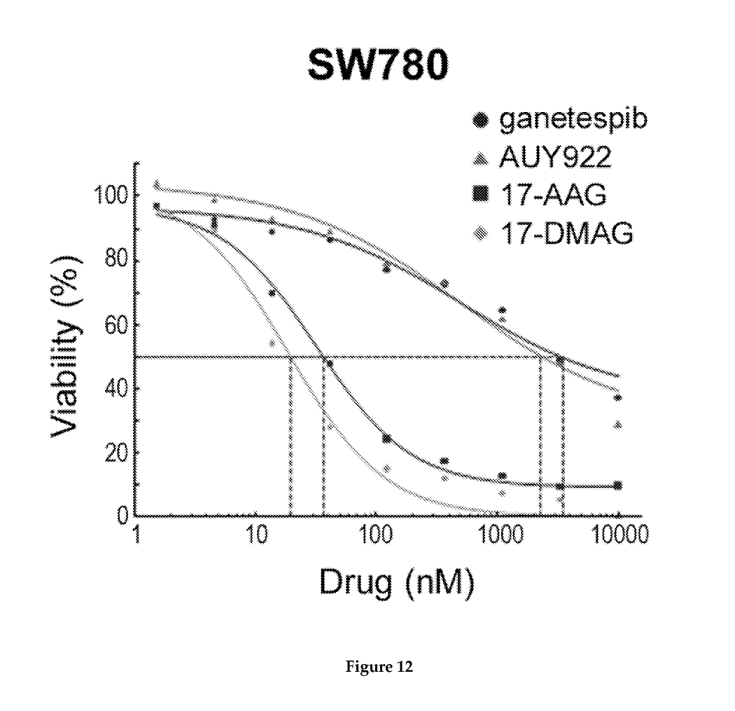

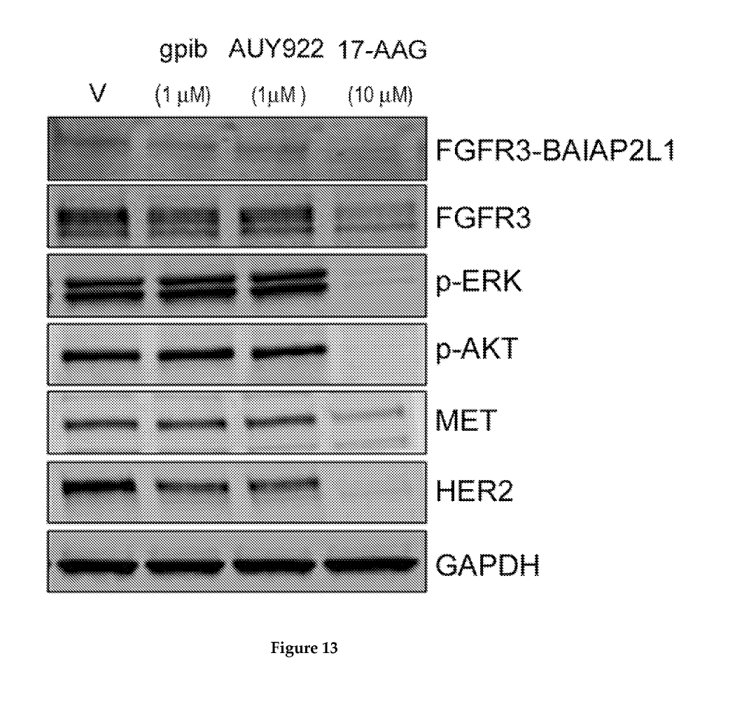

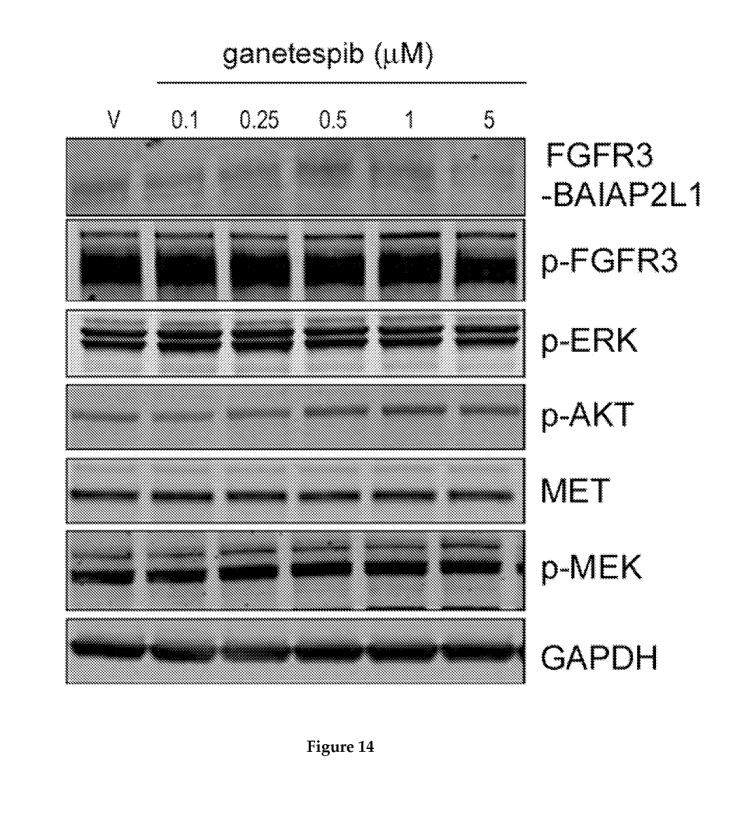

This pattern was repeated in the SW780 line which harbors an FGFR3-BAI1AP2L1 gene rearrangement. The two resorcinol-based inhibitors were again only weakly cytotoxic, yet the cells remained acutely sensitive to 17-AAG and 17-DMAG treatment (FIG. 12). In stark contrast to 17-AAG, neither ganetespib nor AUY922 were found to significantly affect FGFR3-BAI1AP2L1 fusion protein levels, wild-type FGFR3 downstream effector pathways (p-ERK, p-AKT), or other established HSP90 client proteins (MET, HER2) (FIG. 13). These data were additionally supported by the dose-dependent analysis of ganetespib activity shown in FIG. 14. No changes in FGFR3-BAI1AP2L1, phosphorylated FGFR3, ERK, AKT or MEK, or MET levels were observed, suggesting that the HSP90 inhibitory activity of the compound was compromised in this cell line. Taken together, these data show that the FGFR3-TAAC3 and FGFR3-BAI1AP2L1 gene fusion products were sensitive HSP90 client proteins, as evidenced by the potent effects of 17-AAG and 17-DMAG on protein destabilization and cell viability in RT4 and SW780 cells, respectively. However, the lack of client protein modulation and cytotoxic activity seen with ganetespib and AUY922 suggested that these two cell lines exhibited an intrinsic level of resistance to the resorcinol based, second-generation HSP90i.

### Example 5

### High Expression of UGT1A9 in FGFR3 Fusion-Positive Bladder Cells Results in Glucuronidation and Efflux of Ganetespib

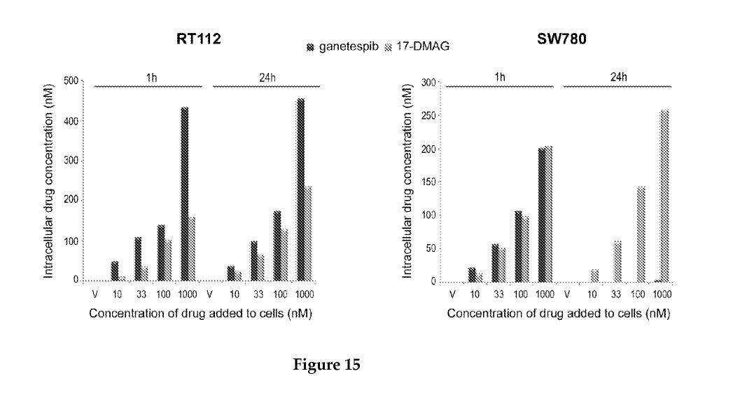

To examine the mechanisms underlying the differential sensitivities to specific HSP90i exhibited by the FGFR3 fusion-positive cell lines, comparison was made of the intracellular concentrations of ganetespib and 17-DMAG in the ‘resorcinol-sensitive’ RT112 and ‘resorcinol-resistant’ SW780 cell lines as a function of time (FIG. 12). In RT112 cells, both compounds accumulated in a dose-dependent manner 1 hour post treatment, albeit with comparatively higher total ganetespib levels than 17-DMAG at each concentration tested. Moreover, the intracellular levels for each HSP90i seen after 1 hour appeared to approach saturation since no significant increases were observed at the 24-hour time point (FIG. 15, left panel). In the SW780 cell line, the initial accumulations of ganetespib and 17-DMAG were indistinguishable, with each HSP90i readily entering and accumulating to near identical concentrations at 1 hour. By 24 hours, levels of 17-DMAG were maintained and modestly increased; in stark contrast virtually no ganetespib was detectable within the intracellular compartment of SW780 (FIG. 15, right panel).

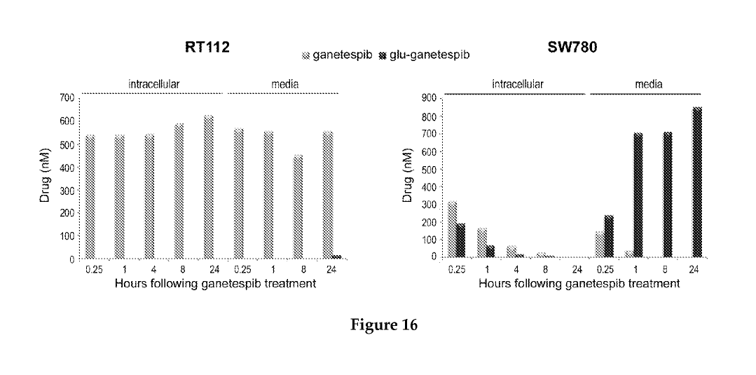

These data suggested that ganetespib resistance was not due to differences in HSP90 biology but likely attributable to drug efflux and/or metabolism mechanisms. Similar to AUY922, ganetespib is susceptible to metabolism by the UGT1A family of UDP-glucuronosyltransferase enzymes that transform small lipophilic molecules into water-soluble, excretable metabolites. Following glucuronidation, the primary metabolites of ganetespib are two glucuronides of the parent compound. Therefore the intracellular and secreted levels of ganetespib and its glucuronidated metabolites were measured in RT112 and SW780 cells as a function of time (FIG. 16). No glucuronidated form of ganetespib was detectable within RT112 cells over a 24-hour time course and only a minor concentration of metabolites was observed in the culture medium at the last time point (FIG. 16, left panel). Notably, approximately 40% of the total intracellular drug levels present in the SW780 line 15 minutes following drug addition were glucuronidated compounds (FIG. 16, right panel), indicating that ganetespib was being rapidly metabolized in these cells. In agreement with the data shown in FIG. 15, intracellular SW780 ganetespib concentrations decreased to undetectable levels by 24 hours. This was concomitant with a marked accumulation of metabolite secretion into the culture media with time (FIG. 16, right panel).

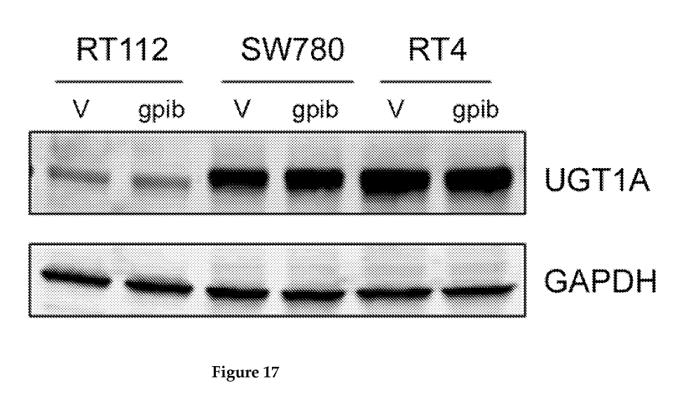

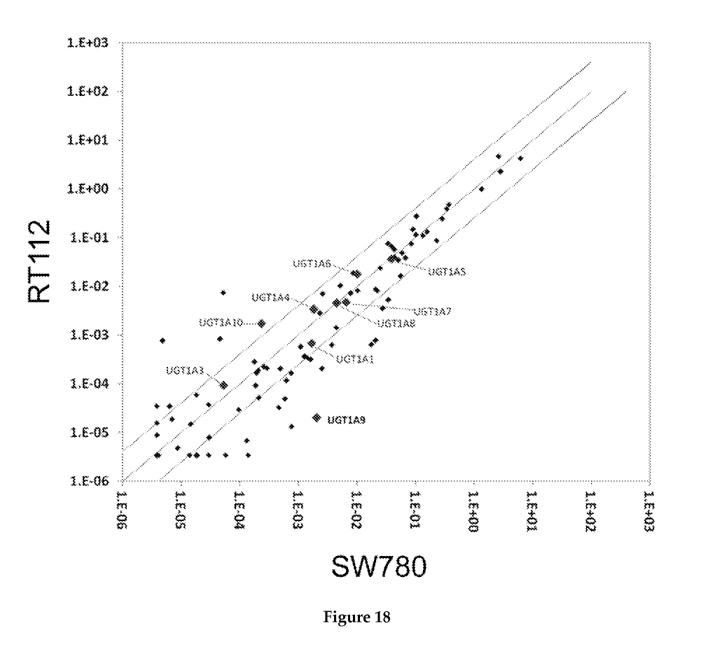

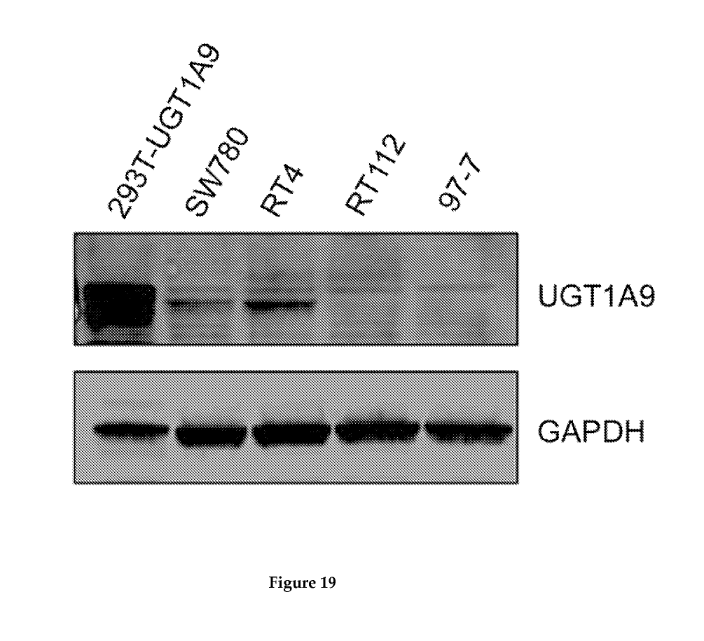

When overall UGTA1 expression was evaluated in the three FGFR3 fusion-positive bladder lines, it was found that both SW780 and RT4 cells expressed considerably higher endogenous levels compared to RT112 (FIG. 17). Microarray analysis was subsequently performed to examine differences in metabolism-related gene expression between the RT112 and SW780 cell lines. Of nine UGTA1 family members identified in that screen, UGT1A9 showed approximately 100-fold higher basal expression in SW780 cells compared to RT112 cells (FIG. 18). To confirm this observation, UGT1A9 protein expression was evaluated in a panel of bladder lines, with UGT1A9-transfected 293T cells serving as a positive control (FIG. 19). The SW780 and RT4 cell lines expressed considerably higher endogenous levels of enzyme compared to RT112, in which UGT1A9 protein was not readily detected. Further, UGT1A9 protein expression was similarly absent in 97-7 cells, which were also sensitive to ganetespib exposure. These data indicated that of the three FGFR3 fusion-positive lines, SW780 and RT4 cells had a higher intrinsic propensity to metabolize ganetespib than RT112, and suggested that this phenotypic variation contributed to the differential sensitivities seen following drug exposure. Taken together, our findings support the premise that high endogenous expression of UGT1A9 in SW780 cells promoted the rapid glucuronidation and subsequent efflux of ganetespib, sufficient to account for its lack of bioactivity in this line.

In summary, these studies provide proof that targeting FGFR3 gene fusions via selective Hsp90 inhibition represents a rational therapeutic strategy for bladder tumors oncogenically driven by this mechanism of aberrant receptor activation. Based on select advantages of this approach over FGFR-specific kinase inhibitors, these findings are likely to provide a framework for the development of new targeted agents and biomarkers designed to improve therapeutic outcomes for bladder cancer patients.

### Example 6

### Expression Levels of UGT1A Vary in Primary CRCs and in CRC-Derived Cell Lines, and Correlate with Resistance to Ganetespib

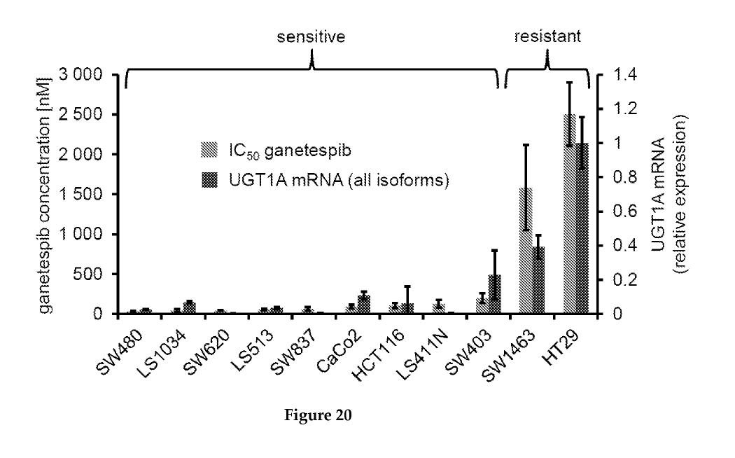

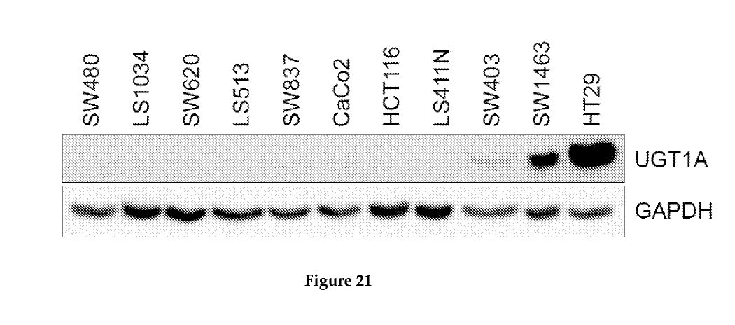

The sensitivity of a panel of eleven CRC-derived cell lines was tested towards ganetespib by analyzing their proliferation over a period of four days through quantitative light microscopy. Drug concentrations that inhibited the growth rate by 50% were determined and found to vary strongly between cell lines, ranging from 36 nM to 2500 nM (FIG. 20). Comparison was next made for the pattern of ganetespib resistance to the respective whole genome gene expression profiles previously established for these cell lines. A number of genes were found to correlate in their expression with ganetespib resistance. Among them, the UGT1A gene stood out due to its known broad-range drug-metabolizing activity. Assessment was also independently made on the expression levels of UGT1A by quantitative RT-PCR and it's confirmed that it closely correlated with ganetespib resistance (FIG. 20). Of note, HT29 cells and SW1463 cells showed high levels of UGT1A and were highly resistant to (i.e. proliferated despite high concentrations of) ganetespib. Corresponding elevated UGT1A protein levels were detected by immunoblot analysis in these two resistant lines but not in the sensitive cell lines (FIG. 21).

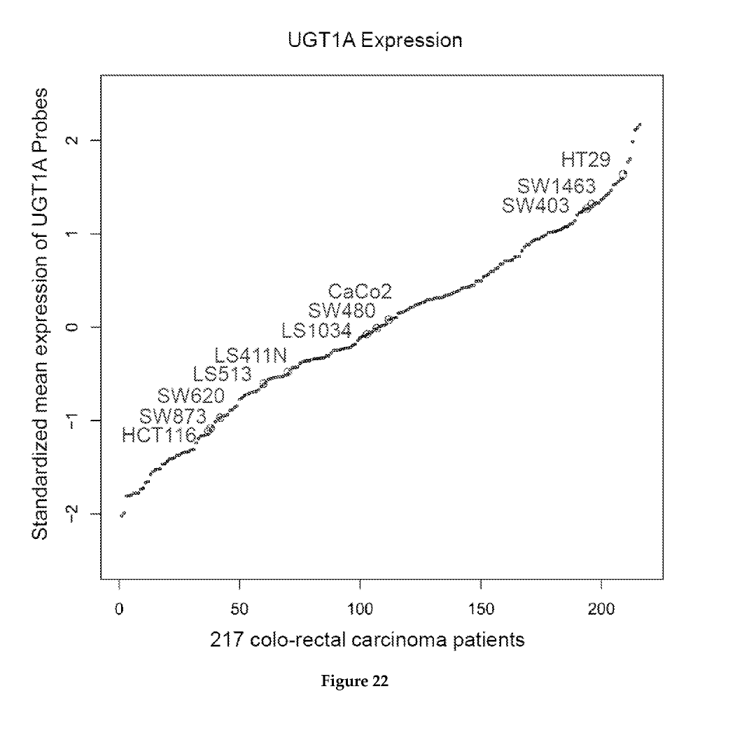

UGT1A is highly expressed in normal colonic tissue, yet from the CRC cell line data the frequency of UGT1A expression was low. Analysis of the UGT1A expression levels from a microarray database of >200 CRC patient tumors revealed that a small but distinct subset of CRC tumors show elevated UGT1A expression that is comparable to the ganetespib resistant cell lines (FIG. 22), suggesting that transformation may select against UGT1A expression in most but not all CRCs.

### Example 7

### Sensitivity to the Hsp90 Inhibitor 17-N-Allylamino-17-Demethoxygeldanamycin (17-AAG) does not Correlate with UGT1A Expression

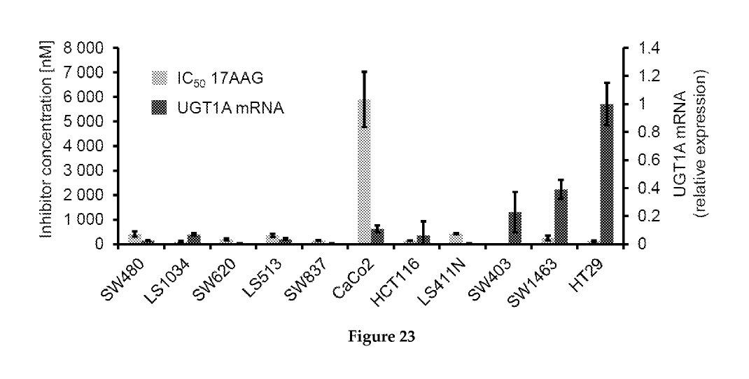

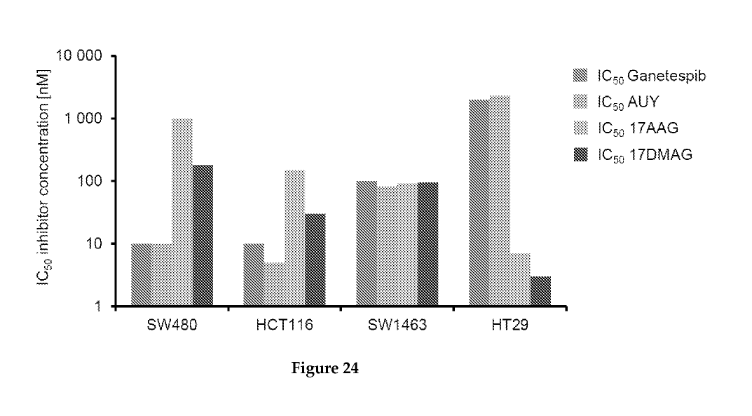

Determination was made on whether the UGT1A expression levels also correlate with the resistance of CRC cells towards a structurally different class of HSP90 inhibitor. As shown in FIG. 23, this was not the case for the Hsp90 inhibitor 17-AAG. Although 17-AAG (17-N-allylamino-17-demethoxygeldanamycin) also showed varying activity against the different cell lines, its sensitivity distribution did not correlate with the expression levels of UGT1A. This comparison revealed that ganetespib- or AUY992-resistant cell lines could be highly sensitive to 17-AAG or its more hydrophilic relative 17-DMAG (17-Dimethylaminoethylamino-17-demethoxygeldanamycin) (for example HT29) or vice versa (for example SW480) (FIG. 24). For 17-AAG and 17-DMGA, it is known that the activation of these prodrugs is facilitated by NAD(P)H:quinone oxidoreductase 1 (NQO1), an oxidoreductase that reduces their quinone moieties. The 17-AAG-resistant cell line CaCo2 was previously reported to possess a polymorphism of the NQO1 gene (the *2 polymorphism), leading to rapid proteasomal degradation of the NQO1 enzyme and hence strongly reduced NQO1 activity, providing a mechanistic basis for their resistance to 17-AAG. Taken together, Hsp90 inhibitors of different chemical classes show distinct activities against a panel of CRC cell lines, arguing that the resistance mechanisms are not directly related to the intrinsic properties of Hsp90 and its clients, but rather result from an upstream phenomenon such as drug stability.

### Example 8

### UGT1A Knockdown Sensitizes Previously Resistant CRC Cells to Ganetespib

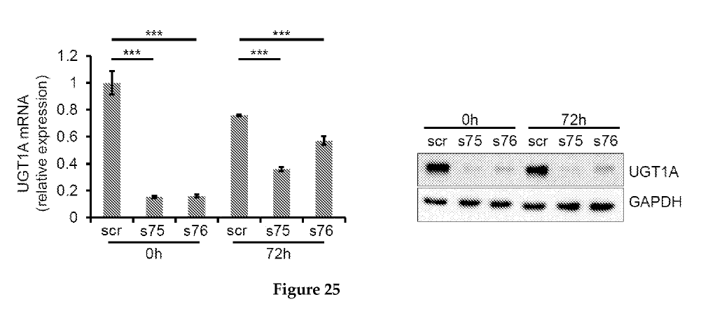

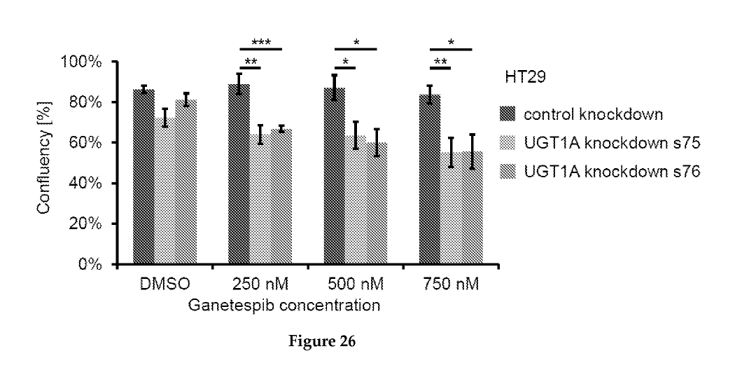

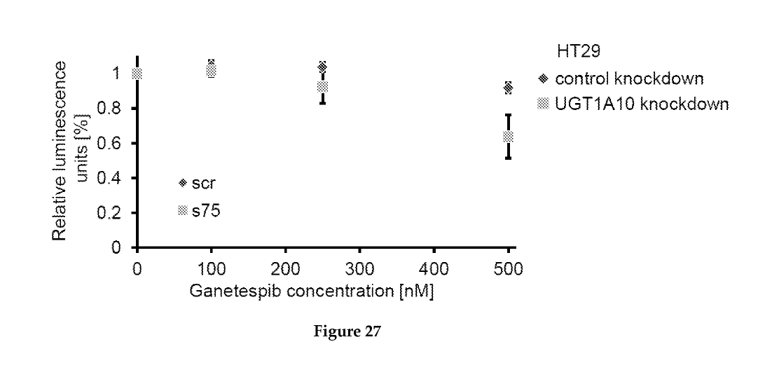

The UGT1A mRNA and protein levels were reduced to determine whether elevated UGT1A expression was directly responsible for ganetespib resistance in the ganetespib-resistant cell line HT29 with two distinct siRNAs (s75 and s76), as validated by quantitative RT-PCR and immunoblot analysis (FIG. 25) and evaluated cell number and viability. From the results, decreased UGT1A expression enhanced sensitivity towards ganetespib treatment, as revealed by reduced cell proliferation (FIG. 26) and reduced cell viability (FIG. 27). These data indicate that elevated UGT1A levels causally contribute to ganetespib resistance.

### Example 9

### Several Isoforms of UGT1A Correlate with Ganetespib Resistance, and Overexpressed UGT1A10 Renders Previously Sensitive Cells Resistant

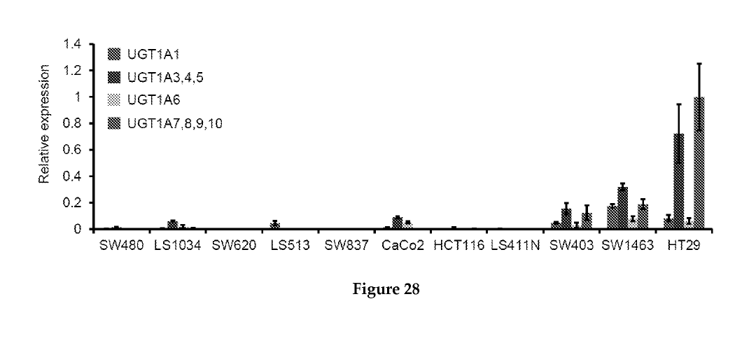

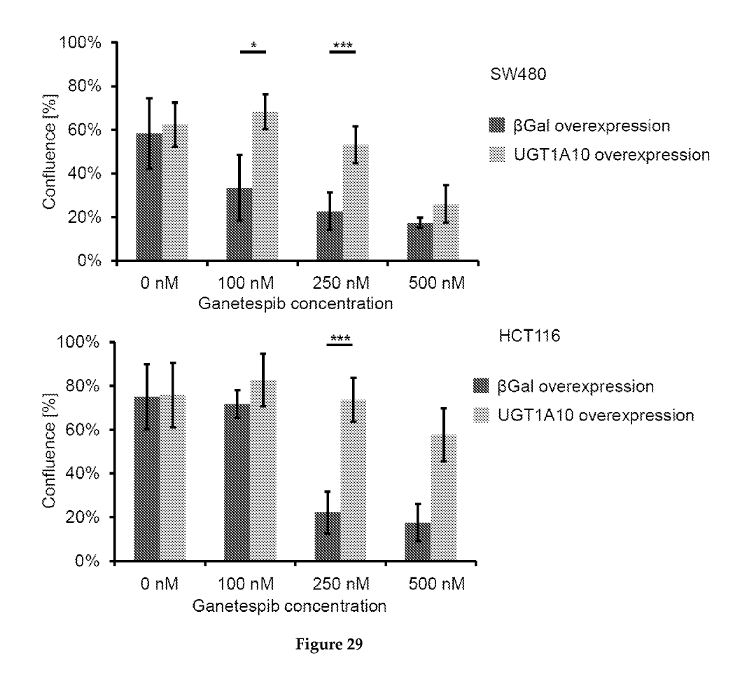

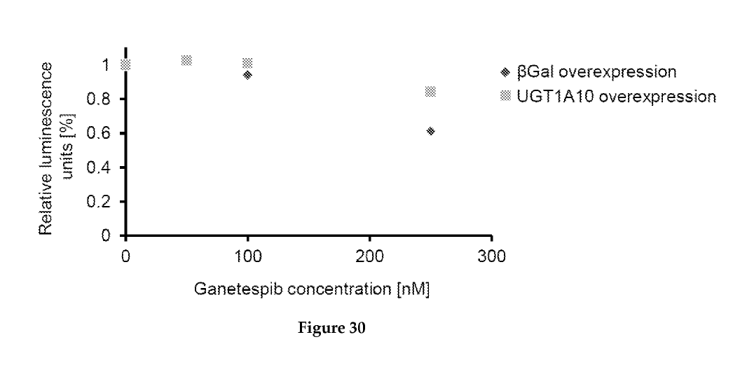

The UGT1A gene can be expressed in at least 9 different but related isoforms, allowing the glucuronyl-conjugation of a broad palette of substrates. To test which isoforms might be responsible for ganetespib resistance, selective RT-PCR assays were done to amplify groups of closely related isoform. This approach had to be taken since some isoforms differ from each other only in a few nucleotide positions, making it virtually impossible to distinguish them by PCR primers. To account for this fact, primers were chosen to amplify groups of UGT1A isoforms based on primer sequences corresponding to UGT1A isoform 1, isoforms 3-5, isoform 6, and isoforms 7-10 (note that isoform 2 does not exist). It was found that the expression levels of isoform cluster 7-10 as well as cluster 3-5 correlated with ganetespib resistance (FIG. 28). It is important to note that ganetespib resistance could simultaneously be conferred by several individual isoforms within a cluster, given the high similarity between them. As proof of principle for the causal role of UGT1A in ganetespib resistance, isoform 10 was chosen as an example. The sensitive cell lines HCT116 and SW480 cells were transfected (both express low endogenous UGT1A levels, cf. FIG. 20) to overexpress UGT1A10. Indeed, this led to increased cell proliferation (FIG. 29) and increased cell viability (FIG. 30) in the presence of ganetespib.

In conclusion, UGT1A expression is not only necessary for maintaining ganetespib resistance, but it is also sufficient to convert a sensitive cell line into a resistant on

### Example 10

### UGT1A-Expressing CRC Cells Fail to Destabilize HSP90 Client Proteins in Response to Ganetespib, but not to 17AAG

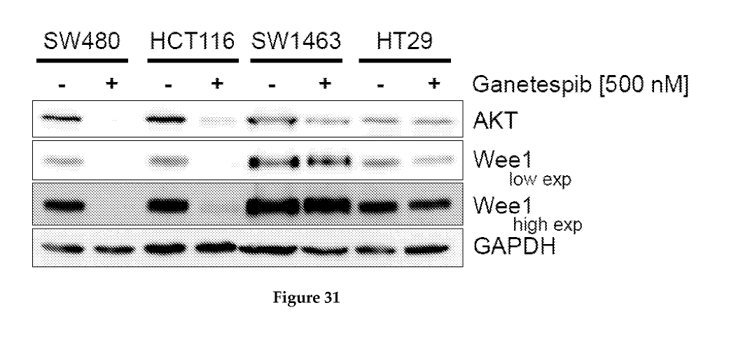

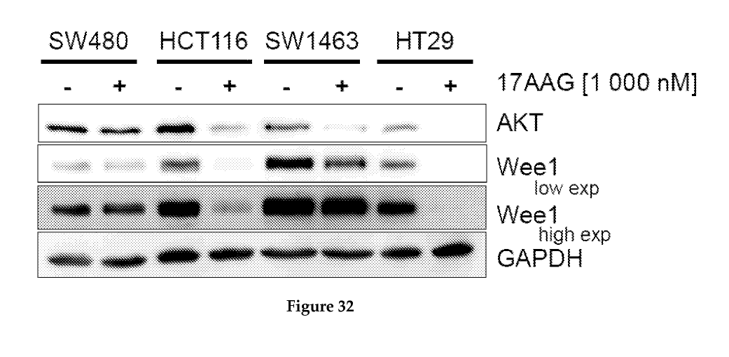

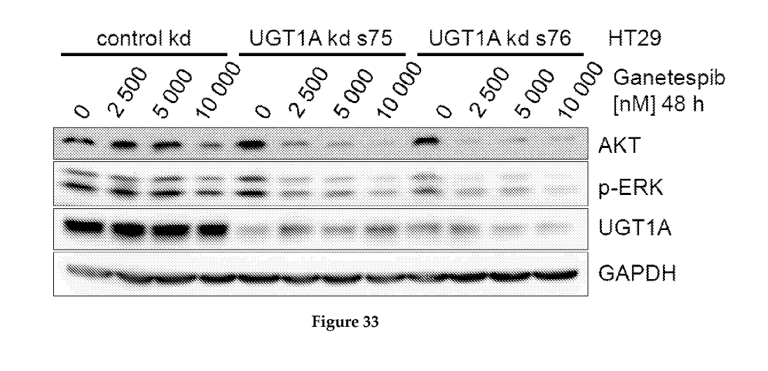

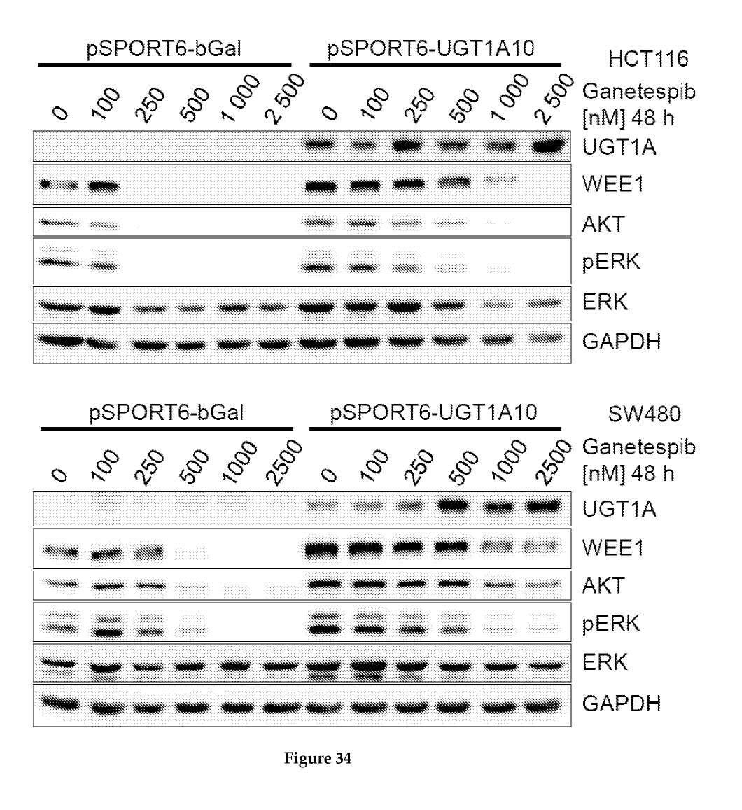

HSP90 stabilizes a number of cancer-driving proteins, and Hsp90 inhibitors are known to decrease the abundance of such Hsp90 clients by enhancing their proteasomal degradation. It was determined to find out whether UGT1A-mediated resistance to ganetespib is also mediated by maintaining the stability of Hsp90 client oncoproteins. Indeed, immunoblot analysis revealed that upon treatment with ganetespib, resistant cells, which expressed high levels of UGT1A (HT29, SW1463, see FIG. 21) failed to decrease the levels of the representative Hsp90 clients Wee1 and AKT. In contrast, sensitive cells, which expressed undetectable levels of UGT1A (SW480, HCT116, see FIG. 21) did destabilize their Hsp90 clients (FIG. 31). In contrast, 17AAG destabilized Hsp90 clients in HT29, SW1463 and HCT116 cells but not in SW480 cells (FIG. 32), exactly reflecting the overall 17AAG resistance pattern (FIGS. 23 and 24). Moreover, in direct support of a causal mechanism, UGT1A knockdown led to destabilization of Hsp90 client proteins in ganetespib-resistant HT29 cells (FIG. 33). Conversely, UGT1A overexpression in originally ganetespib-sensitive HCT116 and SW480 cells stabilized HSP90 clients in the presence of ganetespib (FIG. 34). Taken together, cellular resistance to Hsp90 inhibitors is reflected by the failure of such inhibitors to destabilize Hsp90 client proteins.

### Example 11

### Ganetespib is a Substrate for UDP-Glucuronosyl Conjugation by Tumor Cells

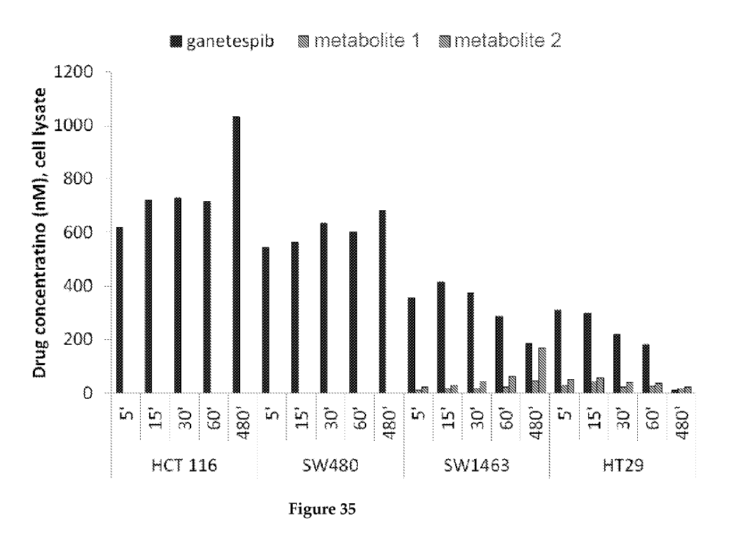

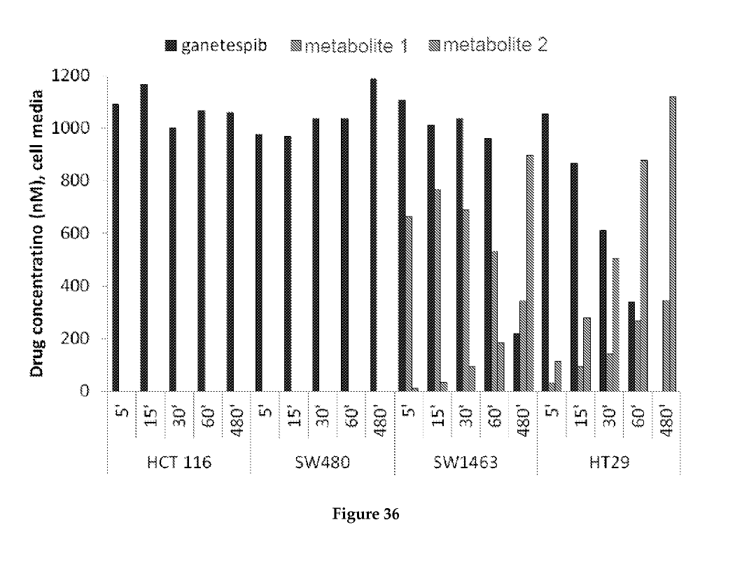

The resistance mediated by UGT1A strongly suggests that UGT1A conjugates ganetespib, leading to its inactivation and excretion from the tumor cells. To this end, measurements were made on the intracellular and secreted levels of ganetespib and its two primary glucuronidated metabolites kinetically over 8 hours in ganetespib sensitive and resistant CRC cell lines (FIGS. 35 and 36). Ganetespib was highly stable in the sensitive cell lines but rapidly metabolized and excreted in the resistant cell lines, evident by a decrease in ganetespib levels and a rise in ganetespib glucuronides. Interestingly, the kinetics for ganetespib metabolism were slower in SW1463 compared to HT29, with ˜190 nM ganetespib present in SW1463 at 8 hr compared to just 13 nM in HT29. This correlated with the level of UGT1A expression in these two lines (FIG. 21) and thus the greater sensitivity of SW1463 cells to ganetespib than HT29 cells (IC50 of 100 nM versus 2000 nM, respectively, based on cell viability; cf. FIG. 24). Therefore, both the presence and level of UGT1A expression may be important indicators of ganetespib activity in CRC.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

1. The method of claim 4, wherein the cancer has a mutation in FGFR3.

2. The method of claim 4, wherein the cancer has a low level expression of UGT1A.

3. The method of claim 4, wherein the cancer has a low level expression of UGT1A9 and/or UGT1A10.

4. A method of treating cancer in a subject, comprising administering ganetespib, or NVP-AUY922 or a pharmaceutically acceptable salt or a tautomer thereof, wherein the cancer has a mutation in FGFR3, and/or a low level expression of UGT1A, and/or with a low level expression of UGT1A9 and/or UGT1A10.

5. The method of claim 8, the method comprising:
(a) obtaining a tissue sample from the cancer of the subject with cancer;
(b) detecting FGFR3 mutations in said sample; and
(c) treating the subject with an effective amount of ganetespib or NVP-AUY922 if the patient has a FGFR3 mutation; and excluding the subject from ganetespib or NVP-AUY922 therapy if the subject does not have a FGFR3 mutation.

6. The method of claim 8, the method comprising:
(a) obtaining a tissue sample from the cancer of the subject with cancer;
(b) determining UGT1A gene expression in said sample; and
(c) treating the subject with an effective amount of ganetespib or NVP-AUY922 if the subject has a low level of UGT1A expression; and excluding the subject from ganetespib or NVP-AUY922 therapy if the subject does not have a low level of UGT1A expression.

7. The method of claim 8, the method comprising:
(a) obtaining a tissue sample from the cancer of the subject with cancer;
(b) determining UGT1A9 and/or UGT1A10 gene expression in said sample; and
(c) treating the subject with an effective amount of ganetespib or NVP-AUY922 if the subject has a low level of UGT1A9 and/or UGT1A10 expression; and excluding the subject from ganetespib or NVP-AUY922 therapy if the subject does not have a low level of UGT1A9 and/or UGT1A10.

8. A method of treating a subject with cancer, the method comprising:
(a) obtaining a tissue sample from the cancer of the subject with cancer;
(b) detecting FGFR3 mutations, and/or UGT1A gene expression, and/or UTUGT1A9 and/or UGT1A10 gene expression in said sample; and
(c) treating the subject with an effective amount of ganetespib or NVP-AUY922 if the subject has a mutation in FGFR3, and/or a low level of UGT1A expression, and/or a low level of UGT1A9 and/or UGT1A10 expression; and excluding the subject from ganetespib or NVP-AUY922 therapy if the subject does not have a FGFR3 mutation, and/or low level of UGT1A expression, and/or a low level of UGT1A9 and/or UGT1A10 expression.

9. The method of claim 8, wherein the subject is treated with an anti-cancer therapy other than ganetespib or NVP-AUY922, if the subject does not have a FGFR3 mutation and/or low level of UGT1A expression, and/or a low level of UGT1A9 and/or UGT1A10 expression.

10. The method of claim 4, wherein the cancer is multiple myeloma, bladder cancer, cervical carcinoma, urothelial carcinoma, colorectal cancer, or another solid tumor.

11. The method of claim 4, wherein the method further comprises administering an FGFR3 inhibitor.

12. The method of claim 11, wherein the FGFR3 inhibitor is BGJ398.

13. The method-of claim 4, wherein the method further comprises administering one or more additional anticancer agents.

14. The method of claim 13, wherein the one or more agents are selected from the group consisting of BEZ-235, AZD6244, AZD8055, SN-38, gemcitabine, camptothecin, docetaxel, cisplatin, oxaliplatin, crizotinib, paclitaxel, trastuzumab, and pemetrexed.

15. The method of claim 4, wherein the amount of ganetespib administered is from about 75 mg/m² to about 260 mg/m².

16. The method of claim 4, wherein the amount of ganetespib administered is from about 125 mg/m² to about 260 mg/m².

17. The method of claim 4, wherein the amount of ganetespib administered is from about 175 mg/m² to about 260 mg/m².

18. The method of claim 4, wherein ganetespib administered is about 75 mg/m², about 85 mg/m², about 100 mg/m², about 110 mg/m², about 115 mg/m², about 120 mg/m², about 145 mg/m², about 150 mg/m², about 175 mg/m², about 180 mg/m², about 200 mg/m², about 215 mg/m² or about 260 mg/m².

19. The method of claim 4, wherein the amount of NVP-AUY922 administered is from about 0.5 mg/kg to about 200 mg/kg.

20. A kit to practice the method of claim 4.


SEQUENCE LISTING (12 sequences)

SEQ ID NO: 1 - DNA, 20 bp, Artificial Sequence
  source: /note=“Description of Artificial Sequence Synthetic primer”
atctgcttggtcacccgatg

SEQ ID NO: 2 - DNA, 20 bp, Artificial Sequence
  source: /note=“Description of Artificial Sequence Synthetic primer”
tccatgcgctttgcattgtc

SEQ ID NO: 3 - DNA, 22 bp, Artificial Sequence
  source: /note=“Description of Artificial Sequence Synthetic primer”
gccattccaaagggaggatgtg

SEQ ID NO: 4 - DNA, 21 bp, Artificial Sequence
  source: /note=“Description of Artificial Sequence Synthetic primer”
tgggaacagccagacaaaagc

SEQ ID NO: 5 - DNA, 22 bp, Artificial Sequence
  source: /note=“Description of Artificial Sequence Synthetic primer”
cataatgaggccctgatcaggc

SEQ ID NO: 6 - DNA, 21 bp, Artificial Sequence
  source: /note=“Description of Artificial Sequence Synthetic primer”
aatcgacaggtacttagccag

SEQ ID NO: 7 - DNA, 18 bp, Artificial Sequence
  source: /note=“Description of Artificial Sequence Synthetic primer”
gctggtggtccctcagga

SEQ ID NO: 8 - DNA, 22 bp, Artificial Sequence
  source: /note=“Description of Artificial Sequence Synthetic primer”
cagctcttcttggtcatacggc

SEQ ID NO: 9 - DNA, 18 bp, Artificial Sequence
  source: /note=“Description of Artificial Sequence Synthetic primer”
cacagtgccctgctcctc

SEQ ID NO: 10 - DNA, 26 bp, Artificial Sequence
  source: /note=“Description of Artificial Sequence Synthetic primer”
gtttggagaatttcagaggctatttc

SEQ ID NO: 11 - DNA, 20 bp, Artificial Sequence
  source: /note=“Description of Artificial Sequence Synthetic primer”
atgctgaggatttggaaagg

SEQ ID NO: 12 - DNA, 21 bp, Artificial Sequence
  source: /note=“Description of Artificial Sequence Synthetic primer”
tcatcacatctcgagcaagac